(19) United States
(12) Patent Application Publication
(10) Pub. No.: US 2012/0149724 A1
(43) Pub. Date: Jun. 14, 2012

(54) MODULATING ENDOGENOUS BETA-ENDORPHIN LEVELS

(75) Inventors: David E. Fisher, Newton, MA (US); Gillian L. Fell, Boston, MA (US)

(73) Assignee: THE GENERAL HOSPITAL CORPORATION, Boston, MA (US)

(21) Appl. No.: 13/376,063

(22) PCT Filed: Jun. 3, 2010
(86) PCT No.: PCT/US2010/037182

§ 371 Date: Mar. 2, 2012

Related U.S. Application Data
(60) Provisional application No. 61/184,067, filed on Jun. 4, 2009.

(51) Int. Cl. A61K 31/439; A61P 25/00

(52) U.S. Cl. 514/282

(57) ABSTRACT

Systemic beta-endorphin can be elevated in response to cutaneous irradiation, including ultraviolet and ionizing radiation. Increases in systemic beta-endorphin levels associated with cutaneous irradiation can be modulated with opiate receptor antagonists, particularly compounds that antagonize opioid receptor binding by beta endorphin.

### CLAIM OF PRIORITY

This application claims priority under 35 USC §119(e) to U.S. Patent Application Ser. No. 61/184,067, filed on Jun. 4, 2009, the entire contents of which are hereby incorporated by reference.

### TECHNICAL FIELD

This disclosure relates to the modulation of systemic beta endorphin levels, including changes in systemic beta endorphin concentration associated with cutaneous irradiation.

### BACKGROUND

Cutaneous irradiation induces biological effects depending on the particular wavelength of the radiation. Radiation in the UVA region mainly causes damage, such as photo-aging, to the skin. Ionizing radiation (e.g., gamma-rays, X-rays, UV-irradiation, microwaves, electronic emissions, or electrons; protons, neutrons, alpha particles, and beta particulate radiation) can be used in radiation therapy to selectively induce DNA damage in tumor cells. Radiation therapy is based on the principle that high-dose radiation delivered to a target area will preferentially kill dividing cells, and thus be more toxic to rapidly dividing tumor cells than to normal cells. However, both excessive UV exposure (e.g., to sunlight and/or artificial tanning facilities) and radiation-induced side effects can negatively impact the quality of life of the individual.

Fatigue associated with cutaneous ionizing irradiation is a common side effect among cancer patients receiving radiation therapy and is the strongest predictor of poor quality of life among these patients (Hickok, J. T., Morrow, G. R., McDonald, S., and Bellg, A. J. Frequency and Correlates of Fatigue in Lung Cancer Patients Receiving Radiation Therapy: Implications for Management. Journal of Pain and Symptom Management. 1996; 11(6): 370-7). Various reports cite 40%-90% of radiation therapy patients report fatigue, and in most instances it hinders patients in daily activities. Methods of treating irradiation induced fatigue have include recommending exercise regimens for irradiated patients with certain types of cancer, utilizing relaxation techniques, and monitoring sleep quantity and quality. In some instances, cancer patients experiencing treatment-related fatigue can be screened for an etiologic cause of fatigue that can be treated. For example, if a cancer treatment causes anemia resulting in fatigue, the patients can be treated for anemia with agents that increase the production of red blood cells (e.g., erythropoietin). However, these methods have been largely non-specific in treating the causes of irradiation-associated fatigue in patients, with varied effectiveness in patients.

Excessive UV exposure (e.g., indoor or outdoor tanning two or more times per week) is associated with increased incidence of skin cancer. Systemic effects of cutaneous radiation exposure (e.g., sun-seeking and radiation-induced fatigue) are believed to include psychological or emotional reactions. Methods of reducing UV-seeking behavior have focused on educational measures to raise awareness of UV-associated skin cancer risk.

There remains a need to identify improved methods of treating negative biological effects of cutaneous irradiation, including UV- and ionizing-irradiation. For example, no known method of treating sun-seeking behavior or radiation-induced fatigue are based on specific pharmacologically treatable physiologic causes. Reports have disclosed that rats exposed to high doses of total-body gamma radiation become lethargic and hypokinetic in a way that mimics their response to morphine. For example, one study showed that tolerance to morphine induced by chronic morphine treatment protected rats from radiation-induced lethargy (Mickley, G. A., Stevens, K. E., Burrows, J. M., White, G. A., and Gibbs, G. L. Morphine Tolerance Offers Protection from Radiogenic Performance Deficits. Radiation Research. 1983; 93(2): 381-7). While this report suggests an opioid-mediated mechanism for radiation-induced lethargy, other reports have suggested that neither UV exposure nor ionizing irradiation result in systemic increases in endogenous opioid levels, such as beta-endorphin. (See, e.g., Gambichler, T., et al., “Plasma Levels of Opioid Peptides After Sunbed Exposures,” British Journal of Dermatology, 147: 1207-1211 (2002); Wintzen, M., et al., “Total Body Exposure to Ultraviolet Radiation Does Not Influence Plasma Levels of Immunoreactive Beta-endorphin in Man. Photodermatology,” Photoimmunology, & Photomedicine; 17(6): 256-60 (2001); and Kaur, M., et al., “Plasma Beta-endorphin Levels in Frequent and Infrequent Tanners Before and After Ultraviolet and Non-ultraviolet Stimuli,” Journal of the American Academy of Dermatology; 54(5): 919-920 (2006)). Accordingly, there remains a need to identify methods of treatment relating to cutaneous irradiation, such as mediating irradiation-induced fatigue or excessive UV exposure seeking behavior.

### SUMMARY

This disclosure provides methods and compositions for mediating changes in endogenous beta-endorphin levels resulting from cutaneous irradiation. The invention is based on the discovery that systemic beta-endorphin can be elevated in response to cutaneous irradiation, including ultraviolet and ionizing radiation. Increases in systemic beta-endorphin levels associated with cutaneous irradiation can be modulated with opiate receptor antagonists, particularly compounds that antagonize opioid receptor binding by beta endorphin. Accordingly, methods of treating conditions such as fatigue or depression resulting at least in part from cutaneous irradiation can include the administration of agents that modulate some portion of an opiate path within a subject.

Methods of treating or preventing a medical condition, e.g., fatigue, associated with exposure to cutaneous irradiation can include identifying a subject who has been or will be exposed to cutaneous irradiation, e.g., gamma and ultraviolet cutaneous irradiation, and administering to the subject a therapeutically effective amount of an opioid antagonist to treat or prevent fatigue. A therapeutically effective amount of an opioid antagonist can reduce an elevation of beta-endorphin level in the blood of the subject induced by the cutaneous irradiation.

Also provided are methods of treating or preventing fatigue associated with administration of a chemotherapeutic agent in a subject. The methods can include identifying a subject who has been or will be administered a chemotherapeutic agent, and administering to the subject a therapeutically effective amount of an opioid antagonist to treat or prevent fatigue associated with administration of a chemotherapeutic agent. Preventing fatigue associated with exposure to cutaneous irradiation or administration of a chemotherapeutic agent does not require that fatigue is completely (e.g., 100%) eliminated, only that fatigue or the risk for fatigue is reduced.

Methods of identifying compounds that mediate the opioid receptors or opioid receptor binding compounds are also described. For example, a method can include cutaneously irradiating a subject (e.g., a mouse) a first time and measuring an opioid-mediated phenotype of behavior in the subject after the first cutaneous irradiation, administering a compound to the subject and determining whether the compound mediates an opioid receptor or opioid receptor binding compound in the subject by subsequently observing changes in the opioid-mediated phenotype and/or behavior in the subject. The method can further include cutaneously irradiating the subject a second time and measuring the opioid-mediated phenotype or behavior in the subject after the second cutaneous irradiation, and comparing the opioid-mediated phenotype or behavior in the subject after the second irradiation to the opioid-mediated phenotype or behavior in the subject after the first irradiation.

Compositions for moderating changes in beta-endorphin effect associated with cutaneous irradiation can include a radiation (e.g., ultraviolet) absorbing compound and an opioid antagonist. For example, the composition can be a sun screen lotion, and can be formulated for transdermal delivery of the opioid antagonist.

### BRIEF DESCRIPTION OF THE DRAWINGS

FIGS. 1A and 1B are graphs showing the effects of a single total-body gamma-radiation exposure on plasma beta-endorphin levels.

FIGS. 2A, 2B, and 2C are graphs showing the fluctuations in plasma beta-endorphin concentrations in mice treated with gamma radiation to the tail.

FIGS. 3A, 3B, 3C, and 3D are graphs showing Straub Tail scores over a regimen of tail radiation or mock exposure.

FIGS. 4A, 4B, and 4C show naloxone-reversible changes in analgesic threshold over a regimen of tail radiation or mock exposure.

FIG. 5 is a graph showing the effects of a single dose of UVB exposure on plasma beta-endorphin levels.

FIGS. 6A and 6B are graphs showing the changes in analgesic thresholds in mice following a single UVB exposure.

FIGS. 7A, 7B and 7C are bar graphs showing the changes in pain thresholds and plasma beta-endorphin over a 6-week regimen of chronic low-dose exposure to UVB.

FIG. 8 is a graph showing basal beta-endorphin levels in different strains of C57BL/6 mice.

FIG. 9 is a graph showing naloxone-induced somatic symptoms of opiate withdrawal in mice following daily UV exposure or mock exposure.

FIGS. 10A and 10B are graphs showing radiation-induced increases in systemic beta-endorphin with parallel development of lethargy/fatigue in rats as measured by locomotor activity.

### DETAILED DESCRIPTION

The term “ionizing radiation,” as used herein, refer to energy sources that induce DNA damage, such as gamma-rays, X-rays, UV-irradiation, microwaves, electronic emissions, particulate radiation (e.g., electrons; protons, neutrons, alpha particles, and beta particles), and the like. An irradiating energy source may be carried in waves or a stream of particles or photons. Further, an irradiating energy source has sufficient energy or can produce sufficient energy via nuclear interactions to produce ionization (gain or loss of electrons). Ionizing radiation can be directed at target tissues (e.g., a cancer cell population) for purposes of reducing the viability of such tissues. Ionizing radiation can be delivered from an external source or from an internal implant at the site of the target tissue. When using X-ray, clinically relevant doses are preferred, and these may be applied in single doses or fractionated, as is known in the art.

The terms “gray” or “Gy” refer to a unit of measurement for the amount of ionizing radiation energy absorbed by body tissues. A gray is equal to 100 rad and is now the unit of dose. A “centigray” or “cGy” is equal to 1 rad.

The ultraviolet region (UV region) is a region of the electromagnetic spectrum adjacent to the low end of the visible spectrum. The UV region extends between 100-400 nm, and is divided into 3 sub regions: the UVA region (320-400 nm), the UVB region (280-320 nm), and the UVC region (100-280 nm).

The term “minimal erythemal dose” (MED) refers to a quantity of radiation associated with the erythemal potential due to exposure to UV radiation. An MED is defined as the radiant exposure of the UV radiation that produces a just noticeable erythema on previously unexposed skin. The radiant exposure to monochromatic radiation at around 300 nm with the maximum spectral efficacy, which is required for erythema corresponds to an approximate dose of 200 to 2000 J/m², depending on the skin type (i.e., fair vs. dark skin).

The term “cutaneous gamma irradiation” refers to gamma irradiation exposure where there is a cutaneous dose administered.

The terms “patient” or “subject” is used throughout the specification to describe an animal, human or non-human, rodent or non-rodent, to whom treatment according to the methods of the present invention is provided. Veterinary and non-veterinary applications are contemplated. The term includes, but is not limited to, birds, reptiles, amphibians, and mammals, e.g., humans, other primates, pigs, rodents such as mice and rats, rabbits, guinea pigs, hamsters, cows, horses, cats, dogs, sheep and goats. Typical patients include humans, farm animals, and domestic pets such as cats and dogs.

### Cutaneous Ionizing Irradiation

Methods of treating fatigue associated with cutaneous ionizing irradiation are provided. The methods can include administering an opioid antagonist to a subject exposed to cutaneous irradiation with, e.g., gamma irradiation in an amount effective to counteract the effect of elevations of beta-endorphin levels in the blood of the subject, for example to mitigate or prevent a medical condition (e.g., irradiation-induced fatigue) in the subject associated with the cutaneous irradiation.

Oncologic radiation therapy is based on the principle that high-dose radiation delivered to a target area will preferentially kill dividing cells, and thus be more toxic to rapidly dividing tumor cells than to normal cells. Subjects can be animal or human and can be exposed to medically appropriate ionizing radiation. The ionizing radiation is generally administered from an external source configured to deliver a substantial radiation dose to the skin of a subject, and is of a type used in cancer therapy and is intended to destroy or inhibit the ability of tumor cells to divide.

The usual course of oncologic ionizing radiation treatment can include administering therapeutically effective doses of IR once per day, five days a week for several weeks. For example, radiation therapy can last from 2 to 10 weeks, depending on the type of cancer. Each session generally lasts an hour or less. A breast cancer radiation therapy course is typically 6 weeks, with radiation exposure 5 days per week. For curative radiation therapy, the subject can be exposed to a total dose of about 50 Gy to 60 Gy over the therapy course, or an average of 2 Gy per day, for solid epithelial tumors. For lymphomas, the total dose is about 20 Gy to 40 Gy over the therapy course. For adjuvant therapy, the dose is typically about 45 Gy to 60 Gy total dose over the therapy course, with doses of about 1.8 Gy to 2 Gy per day. For example, ionizing radiation therapy can provide small doses of radiation each day that enable healthy cells to repair damage, while rendering cancer cells inactive. The ionizing radiation can be delivered at a rate of 0.25 Gy per minute or higher. The ionizing radiation may be in a total dose of 1.8-2.8 Gy in one day, or in fractions in different days in the range of 0.1 to 2.8 Gy per day and up to 30 days.

Methods to administer radiation are well known in the art. The cutaneous irradiation is administered at a greater dose of irradiation to a cutaneous surface than underlying tissue, as the radiation loses energy as it penetrates through the dermal surface. Exemplary methods include, but are not limited to, external beam radiation, internal beam radiation, and radiopharmaceuticals. For example, radiation can be photon treatment, 3D conformal radiation therapy, intensity modulated radiation therapy, stereotactic radiotherapy, proton beam therapy, brachytherapy, radioimmunotherapy, CyberKnife®, an external beam treatment, or internal radiation treatment. In external beam radiation, subjects are typically exposed to either X-rays or gamma rays. A linear accelerator can be used to deliver high-energy X-rays to the area of the body affected by cancer. External beam radiation, in which the radiation source is outside the body, can be used to treat large areas of the body with a uniform dose of radiation. Internal radiation therapy, also known as brachytherapy, involves delivery of a high dose of radiation to a specific site in the body. The two main types of internal radiation therapy include interstitial radiation, wherein a source of radiation is placed in the effected tissue, and intracavity radiation, wherein the source of radiation is placed in an internal body cavity a short distance from the affected area. Radioactive material may also be delivered to tumor cells by attachment to tumor-specific antibodies. The radioactive material used in internal radiation therapy is typically contained in a small capsule, pellet, wire, tube, or implant. In contrast, radiopharmaceuticals are unsealed sources of radiation that may be given orally, intravenously or directly into a body cavity. Ionizing radiation can be delivered by coupling of radioactive isotopes to delivery molecules.

The ionizing radiation may be directed to a tumor by any guidance procedure, typically involving computed tomography (or CT) images taken shortly before treatment. The patient's body can be marked on the skin to indicate where the radiation should be directed. The patient may be positioned to lie in a body mold as an extra measure to ensure the tumor is in the location indicated by the earlier CT scans. A margin around the tumor is included in the radiation target area to avoid missing any part of a tumor.

Radiation-induced side effects, including radiation-induced fatigue, can significantly impact the quality of life of the patient and may dramatically influence patient compliance with treatment. For example, patients undergoing cutaneous irradiation can experience radiation-induced fatigue. For example, patients may begin to experience fatigue 3-4 weeks into a 6-8-week regimen, and the fatigue can last for days, weeks, or months following the end of the regimen, and in rare cases longer (Back, M., Ahern, V., Delaney, G., Graham, P., Steigler, A., and Wratten, C. Absence of Adverse Early Quality of Life Outcomes of Radiation Therapy in Breast Conservation Therapy for Early Breast Cancer. Australasian Radiology. 2005; 49(1): 39-43. Greenberg, D. B., Sawicka, J., Eisenthal, S., and Ross, D. Fatigue Syndrome Due to Localized Radiation. Journal of Pain and Symptom Management. 1992; 7(1): 38-45. Hickok, J. T., Morrow, G. R., Roscoe, J. A., Mustian, K., and Okunieff, P. Occurrence, Severity, and Longitudinal Course of Twelve Common Symptoms in 1129 Consecutive Patients During Radiotherapy for Cancer. Journal of Pain and Symptom Management. 2005; 30(5): 433-42). Severe fatigue can be experienced equally as often in patients receiving tangential field radiation that is minimally penetrating, often contacting only skin and subcutaneous tissue as in patients receiving deeper radiation. In fact, fatigue incidence and severity have been observed to be more correlated with the dose and surface area of the radiation field rather than the depth (Back, M., Ahern, V., Delaney, G., Graham, P., Steigler, A., and Wratten, C. Absence of Adverse Early Quality of Life Outcomes of Radiation Therapy in Breast Conservation Therapy for Early Breast Cancer. Australasian Radiology. 2005; 49(1): 39-43. Hickok, J. T., Morrow, G. R., Roscoe, J. A., Mustian, K., and Okunieff, P. Occurrence, Severity, and Longitudinal Course of Twelve Common Symptoms in 1129 Consecutive Patients During Radiotherapy for Cancer. Journal of Pain and Symptom Management. 2005; 30(5): 433-42. Hofman, M., Ryan, J. L., Figueroa-Moseley, C. D., Jean-Pierre, P., and Morrow, G. R. Cancer-related Fatigue: The Scale of the Problem. Oncologist. 2007; 12(Suppl 1): 4-10. Jereczek-Fossa, B. A., Marsiglia, H. R., and Orecchia, R. Radiotherapy-related Fatigue. Critical Reviews in Oncology/Hematology. 2002; 41(3): 317-25. Schwartz, A. L., Nail, L. M., Chen, S., Meek, P., Barsevick, A. M., King, M. E., and Jones, L. S. Fatigue Patterns Observed in Patients Receiving Chemotherapy and Radiotherapy. Cancer Investigation. 2000; 18(1): 11-9).

Compounds that mediate the effect of a systemically elevated opioid compound can be administered to patients before, during or after a subject receives cutaneous irradiation in a dose effective to increase the level of an opioid in the blood of the subject.

Methods of treating fatigue associated with cutaneous ionizing radiation are provided herein. The methods are based in part on the discovery that irradiation of skin cells can elevate the systemic levels of the opioid β-endorphin. Testing in animal models (e.g., Examples 1-4) demonstrated elevated systemic levels of β-endorphin following cutaneous ionizing radiation exposure. For example, cutaneous ionizing radiation produced naloxone-reversible changes in phenotypes associated with opioid administration in mice undergoing tail-only radiation.

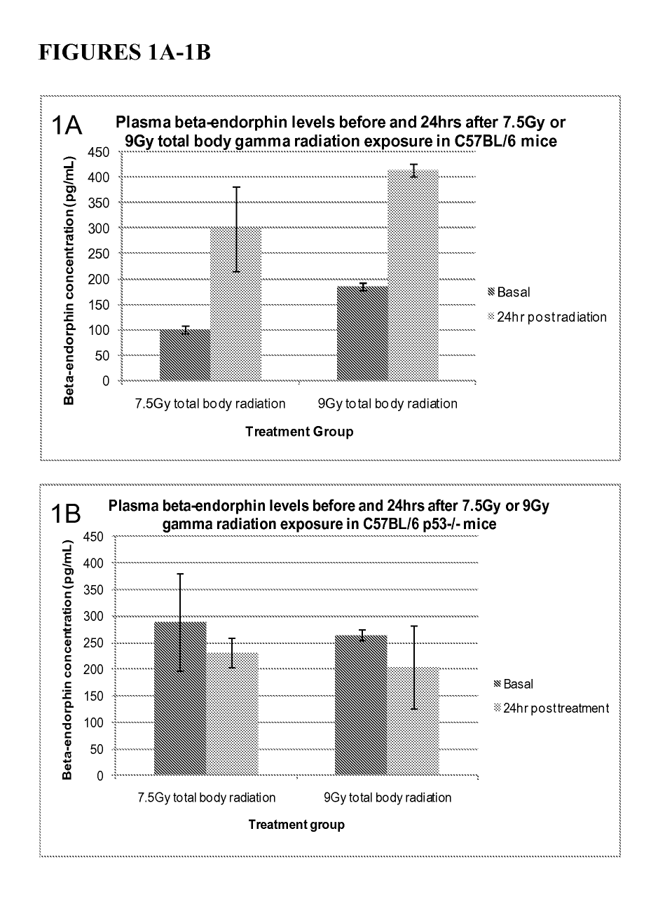

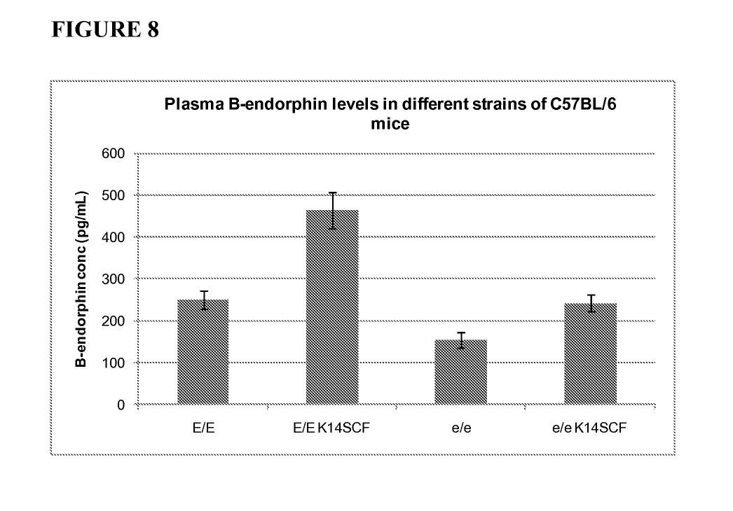

FIGS. 1A-1B are graphs of data showing the elevation of systemic beta-endorphin levels in mice in response to cutaneous ionizing radiation. Wild type C57BL/6 mice were exposed to a single, total body dose of 7.5 Gy or 9 Gy gamma radiation, as described in Example 1. As shown in FIG. 1A, 24 hours following the exposure, results show a tripling of plasma beta-endorphin levels compared to basal values in the mice. To obtain the data shown in FIGS. 1A and 1B, 8-week old mice were exposed to a dose of 7.5 Gy or 9 Gy. Blood was drawn for beta-endorphin measurement by radioimmunoassay prior to and 24 hours after exposure. Data for FIG. 1A was obtained from Wild type mice, and is a compilation of 2 experiments with 5 mice per treatment group per experiment. The graph compares basal to 24 hours post radiation endorphin levels with p<0.05 by t-test for both 7.5 Gy and 9 Gy radiation doses. Data for FIG. 1B was obtained from p53−/−mice, as further described in Example 1, and is a compilation of 2 experiments with 3 mice per treatment group per experiment. The graph in FIG. 1B compares basal to 24 hours post exposure endorphin levels at each dose with p>0.05 by t-test for both 7.5 Gy and 9 Gy. In response to UV exposure, keratinocytes in the skin increase production of the beta-endorphin precursor protein, POMC, in a p53-dependant manner. The data shown in FIG. 1B indicates plasma beta-endorphin elevation following gamma radiation exposure is also p53-dependant. As shown in FIG. 1B, 24 hours following a single, total body dose of 7.5 Gy or 9 Gy gamma radiation, B57BL/6 mice showed no significant changes in plasma beta-endorphin levels compared to baseline values in the same mice.

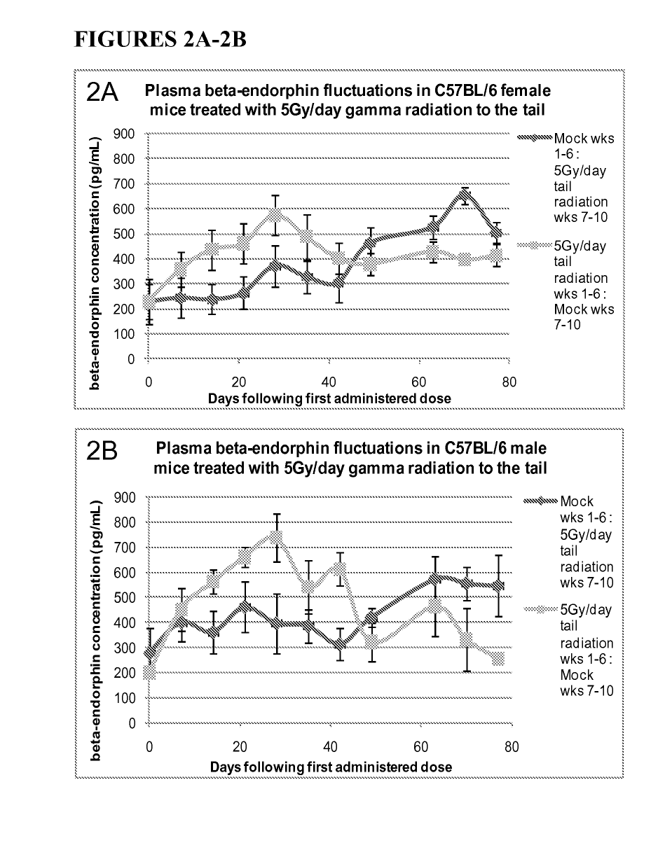

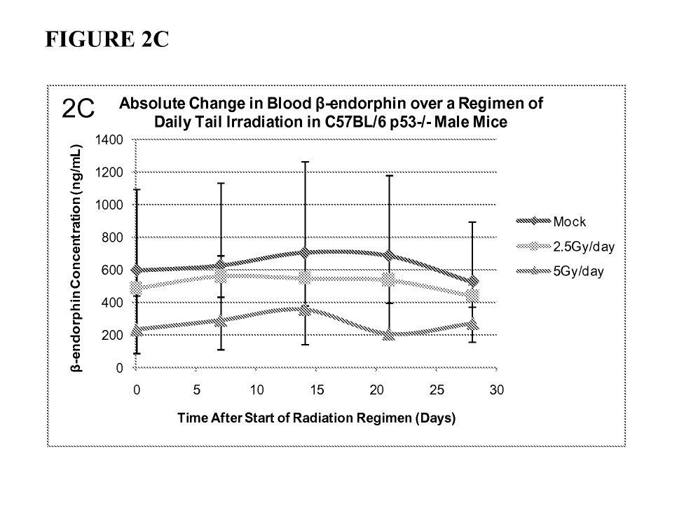

FIGS. 2A and 2B are graphs of experiments described in Example 2, showing that tail-only radiation exposure in mice results in a p53-dependent increase in plasma beta-endorphin levels. Example 2 describes a radiation dosing regimen that mimics a radiation therapy regimen for cancer patients by using low to moderate daily tail radiation. To obtain the data shown in FIGS. 2A and 2B, 8-week old female (FIG. 2A) or male (FIG. 2B) mice were either exposed to a dose of 5 Gy/day gamma radiation or mock exposed for 6 weeks, as further described in Example 2. Data for FIG. 2A was obtained from C57BL/6 female mice, and there were 5 mice per treatment group. Data for FIG. 2B was obtained from C57BL/6 male mice, and there were 5 mice per treatment group, except for the group switching from mock treatment to radiation, there was a loss of 3 mice at week 7. Both male and female mice tested to obtain the data in FIGS. 2A and 2B show a change in systemic beta-endorphin levels in response to daily tail radiation. Mice receiving 5 Gy per day tail radiation for 6 weeks followed by 4 weeks of mock exposure show beta-endorphin levels rising by week 2, peaking at week 4, and begin to dip at week 5. Once the mock exposure began, beta-endorphin levels lowered to a level less than those during the radiation therapy. In mice receiving mock exposure for 6 weeks, the beta-endorphin levels are stable during mock treatment. However, when the mock-exposed mice begin the radiation regimen, beta-endorphin levels rise significantly.

To obtain the data shown in FIG. 2C, mice were either mock exposed or treated with 2.5 Gy/day or 5 Gy/day tail-only radiation over a 3 week course. Beta-endorphin concentrations were measured weekly for 4 weeks. Data for FIG. 2C was obtained from p53−/− mice and there were 3-5 mice per treatment group. FIG. 2C shows data obtained according to Example 2, indicating that the increase in beta-endorphin over a tail radiation regimen is p53-dependent. No significant change in beta-endorphin levels was observed in p53−/− mice when exposed to daily tail radiation. This shows the beta-endorphin elevation seen in wild type mice is a p53-dependent phenomenon. The results of Example 2 suggest that factors in the skin play a causative role, consistent with radiation-induced fatigue observed in patients that are exposed to minimally penetrating radiation.

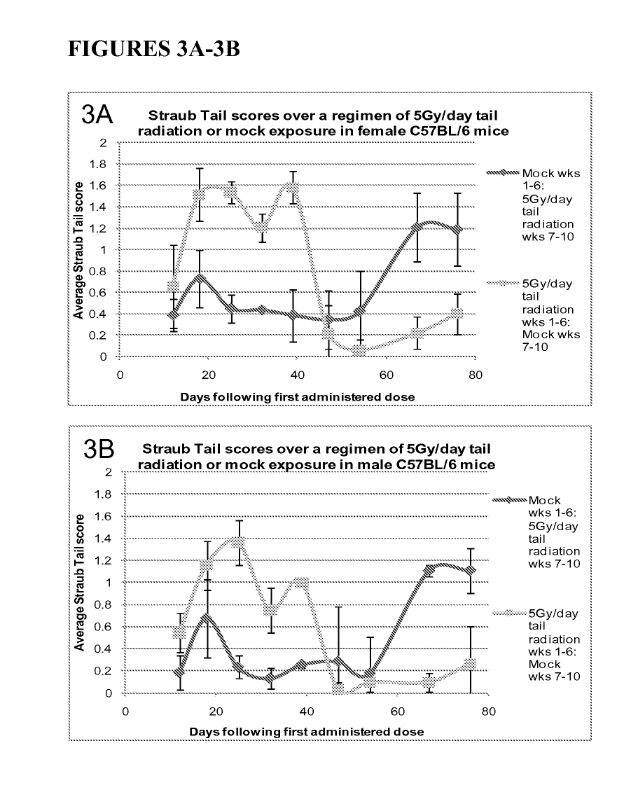

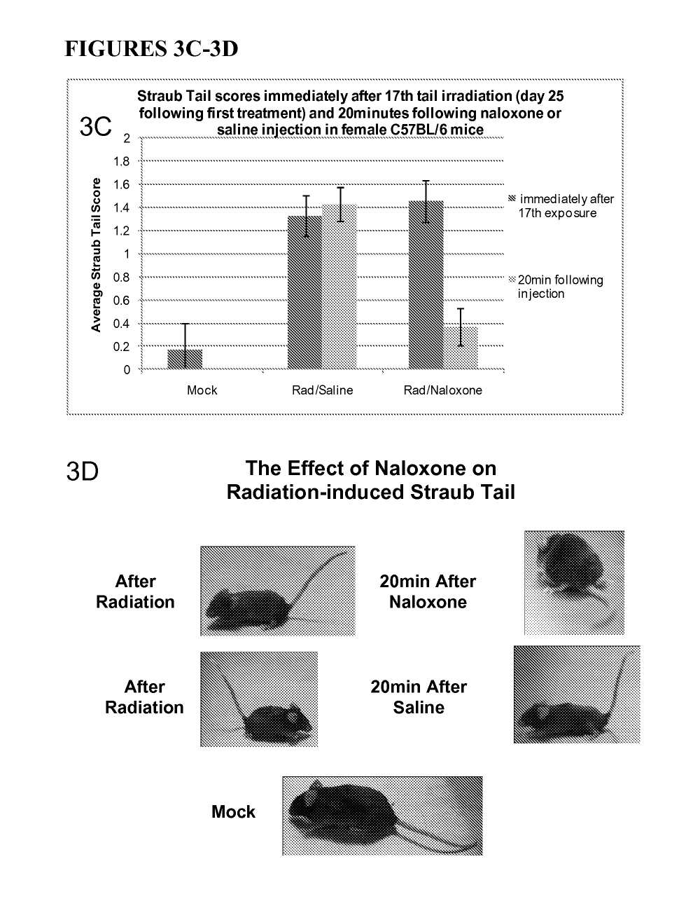

Example 3 describes how the systemic elevation of beta-endorphin is significant enough to cause changes in opioid-mediated phenotypes. Straub Tail in mice is a stiffening and elevation of the tail that results from opioid-mediated contraction of the sacroccygeus dorsalis muscle at the base of the tail. To obtain the data shown in FIGS. 3A and 3B, 8-week old female (FIG. 3A) or male (FIG. 3B) mice were either exposed to a dose of 5 Gy/day gamma radiation or mock exposed for 6 weeks. After 6 weeks, there was a 1 week break, then the treatment groups were switched for 4 weeks, so the 5 Gy/day group was then mock exposed, and the mock exposed group was then exposed to 5 Gy/day. Mice in both groups (FIGS. 3A and 3B) were scored for Straub Tail immediately following tail radiation or mock treatment starting on day 12 of the regimen, 2-4 times per week for the first 6 weeks, then 1 time per week for the final 4 week regimen, after the groups were switched. Data for FIGS. 3A and 3B were obtained from C57BL/6 female and male mice (respectively).

The graph in FIG. 3C shows the effect of naloxone or saline injections on Straub Tail scores. To obtain the data shown in FIG. 3C, after the 17th irradiation dose, the mice were measured for Straub Tail scores and the irradiated mice were divided into two subgroups. The first subgroup was injected with 10 mg/kg naloxone ip. The second subgroup was injected with saline ip. Straub Tail scores were then rescored 20 minutes after the injection. FIG. 3C uses data obtained by Wild type female mice. FIG. 3C shows the opioid antagonist has reversed the Straub Tail, similar to mock-treated levels. The saline injection has no effect on the Straub Tail phenotype.

FIG. 3D shows representative photos of the mice scored in FIG. 3C after the 17th radiation dose. The Straub Tail phenotype occurs in response to tail radiation exposure and returns to basal values following the radiation regimen with kinetics that parallel plasma beta-endorphin fluctuations.

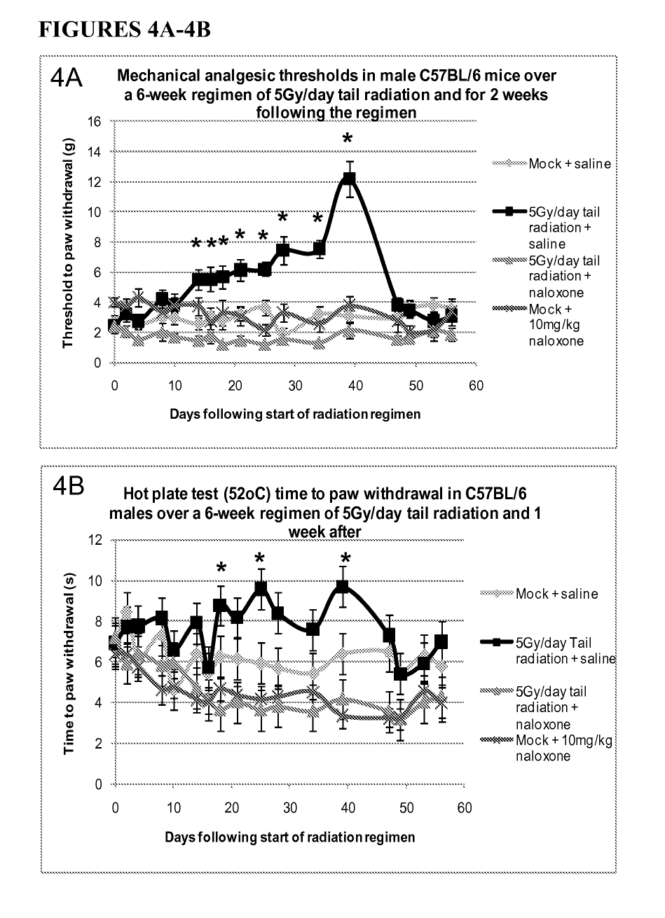

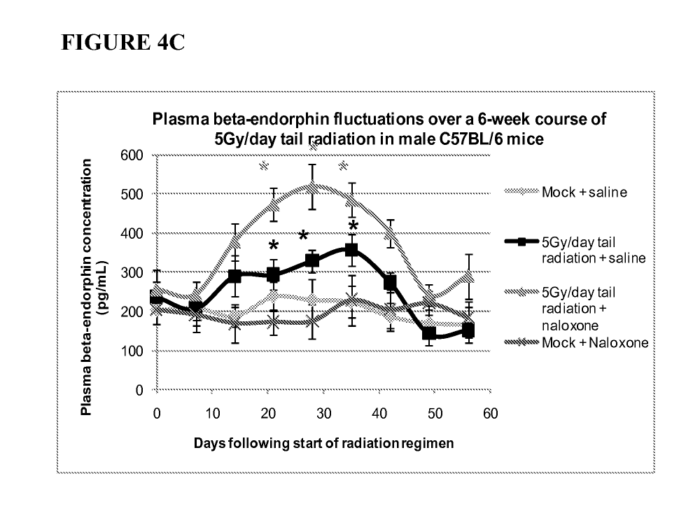

Example 4 describes analgesic response changes in response to tail-only radiation exposure. To obtain the data shown in FIGS. 4A and 4B, 8-week old male mice were either exposed to a dose of 5 Gy/day gamma radiation or mock exposed for 6 weeks. Mechanical (FIG. 4A) and thermal (FIG. 4B) analgesic thresholds were measured twice per week over the course of the regimen, and for 2 weeks after the regimen. The threshold testing was performed in the morning before the radiation treatment or mock exposure. The mock exposed mice were either injected with saline 15 minutes before the testing, or with 10 mg/kg naloxone 15 minutes before the testing. The radiation treatment group was divided into 2 subgroups. The first group was given saline injections 15 minutes before the testing. The second group was given a 10 mg/kg naloxone injection 15 minutes before the testing. For all groups of mice, blood was drawn once per week for beta-endorphin measurement. Data for FIG. 4A was obtained from C57BL/6 male mice, and there were 10 mice per treatment group. Data for FIG. 4B was obtained from C57BL/6 male mice, and there were 10 mice per treatment group. An asterisk indicates p<0.05 by t-test. FIGS. 4A and 4B show both mechanical and thermal analgesic thresholds increase over the radiation regimen similarly to plasma beta-endorphin fluctuations (Figure C).

FIG. 4C illustrates the plasma beta-endorphin fluctuations in the C57BL/6 mice as described in FIGS. 4A and 4B. To obtain the data shown in FIG. 4C, blood was drawn once per week over the 6 week regimen, and for the 2 weeks following. The blood was drawn in the morning prior to the radiation treatment for that day. FIG. 4C uses data obtained by C57BL/6 male mice, 10 mice per treatment group. Mock exposed mice remained stable throughout the regimen. The tail-irradiated mice injected with naloxone show analgesic threshold results similar to the mock exposed mice, suggesting pretreatment with naloxone prevents changes in analgesic thresholds, although beta-endorphin levels rise significantly over the course of the radiation regimen.

Methods of treating irradiation-induced fatigue can include administering a compound that mediates (e.g., reduces or prevents) the opioid receptor binding of opioid compounds present in the blood stream after ionizing radiation. In particular, opioid antagonists can be administered in a dose effective to modulate the physical effects of increased systemic opioid levels induced by cutaneous ionizing irradiation. Opioids are a class of molecules that bind with varying affinities to μ-, δ-, and κ-opioid receptors. The opioid receptors are G-protein coupled receptors that signal primarily through Gαi or Gαq, and are expressed ubiquitously and at varying densities throughout the CNS and periphery. Their effects are complex and lack specificity, as they depend on the target cell type and the ability of the agonist to localize, the degree of specificity of the agonist for a particular receptor subtype (opioids usually have cross-reactivity with multiple receptor subtypes), the subtypes of receptor on the target cell and their densities, the subtypes of adenylyl cyclase expressed in target cells, and the expression or activity of regulators of opioid receptor signaling, such as G-protein receptor kinases (GRKs), which phosphorylate and down-regulate receptor signaling, and β-arrestin, which causes recruitment of clathrin and internalization of the receptor. Within the nervous system, opioids counteract nociceptive inputs and activate reward centers in the brain resulting in a calming, euphoric sensation. This is largely via a μ-receptor/Gαi-mediated mechanism. For this reason, opioids are widely used in the clinical setting to treat pain. However, they carry many undesirable side-effects, including sedation, drowsiness, respiratory depression, constipation, and nausea; mediated by action of these drugs at other receptor subtypes and in other cell types. The rewarding and euphoric effects make opioids reinforcing; thus, they are also considered potentially addictive and can be substances of abuse. In addition, opioid drugs used clinically, such as morphine, can cause fatigue and lethargy.

In one example, methods of treating irradiation-induced fatigue include administering to a patient compounds that antagonize fatigue-inducing effects of systemic beta-endorphin elevation in response to cutaneous ionizing radiation. Beta-endorphin is one of three endogenously-produced opioids, the others being enkephalin and dynorphin. All three share a common N-terminal motif of YGGF(M/L), which is thought to be important for receptor binding. Of them, β-endorphin is the most abundant, with various studies reporting basal levels in human plasma ranging between 1 pM and 12 pM (Batistaki, C., Kostopanagiotou, G., Myrianthefs, P., Dimas, C., Matsota, P., Pandazi, A., and Baltopoulos, G. Effect of Exogenous Catecholamines on Tumor Necrosis Factor Alpha, Interleukin-6, Interleukin-10 and Beta-endorphin Levels Following Severe Trauma. Vascular Pharmacology. 2008; 48(2-3): 85-91. Bender, T., Nagy, G., Barna, I., Tefner, I., Kadas, E., and Geher, P. The Effect of Physical Therapy on Beta-endorphin Levels. European Journal of Applied Physiology. 2007; 100(4): 371-82. Fassoulaki, A., Kostopanagiotou, G., Meletiou, P., Chasiakos, D., and Markantonis, S. No Change in Serum Melatonin, or Plasma Beta-endorphin Levels After Sevoflurane Anesthesia. Journal of Clinical Anesthesia. 2007; 19(2): 120-4. Fassoulaki, A., Paraskeva, A., Kostopanagiotou, G., Tsakalozou, E., and Markantonis, S. Acupressure on the Extra 1 Acupoint: The Effect on Bispectral Index, Serum Melatonin, Plasma Beta-endorphin, and Stress. Anesthesia & Analgesia. 2007; 104(2): 312-7. Fedele, F., Agati, L., Pugliese, M., Cervellini, P., Benedetti, G., Magni, G., and Vitarelli, A. Role of the Central Endogenous Opiate System in Patients with Syndrome X. American Heart Journal. 1998; 136(6): 1003-9. Leppaluoto, J., Westerlund, T., Huttunen, P., Oksa, J., Smolander, J., Dugue, B., and Mikkelsson, M. Effects of Long-term Whole-body Cold Exposures on Plasma Concentrations of ACTH, Beta-endorphin, Cortisol, Catecholamines and Cytokines in Healthy Females. Scandinavian Journal of Clinical and Laboratory Investigation. 2008; 68(2): 145-53). In vitro studies have indicated that β-endorphin is predominantly a μ-receptor agonist, with a km of 9 nM, and has some ability to bind the δ-receptor with a km of 22 nM (Schoffelmeer, A. N., Warden, G., Hogenboom, F., and Mulder, A. H. Beta-endorphin: A Highly Selective Endogenous Opioid Agonist for Presynaptic Mu Opioid Receptors. Journal of Pharmacology and Experimental Therapy. 1991; 258(1): 237-42). Knockout mice have been generated for each element of the opioid system, and have been able to shed some light on the expression patterns and activities of the endogenous opioids (Kieffer, B. L., and Gaveriaux-Ruff, C. Exploring the Opioid System by Gene Knockout. Progress in Neurobiology. 2002; 66(5): 285-306). Centrally, β-endorphin is expressed primarily in the arcuate nucleus of the hypothalamus, where afferent sensory projections are sent to the periaqueductal gray area of the brainstem to temper pain sensation as well as to the nucleus accumbens to enhance calming, positive sensations. In addition, afferent and efferent β-endorphin fibers in the nucleus of the tractus solarius contact the periaqueductal gray, raphe nucleus, and spinal cord (Rubinstein, M., Mogil, J. S., Japon, M., Chan, E. C., Allen, R. G., and Low, M. J. Absence of Opioid Stress-induced Analgesia in Mice Lacking Beta-endorphin by Site-directed Mutagenesis. Proceedings of the National Academy of Sciences. USA. 1996; 93(9): 3995-4000). The anterior pituitary is a central source of peripherally circulating β-endorphin, as β-endorphin can cross the blood-brain-barrier from the pituitary via the P-Glycoprotein transporter (King, M., Su, W., Chang, A., Zuckerman, A., and Pasternak, G. W. Transport of Opioids from the Brain to the Periphery by P-glycoprotein: Peripheral Actions of Central Drugs. Nature Neuroscience. 2001; 4(3): 268-74). Other sources of peripheral β-endorphin include various skin cells as well as cells of the immune system, which secrete β-endorphin as an immune mediator at sites of inflammation.

For example, an opioid antagonist can be administered to a patient before, during or after receiving ionizing irradiation. The opioid antagonist can be administered to the subject in any medically appropriate manner effective to reduce beta endorphin receptor binding. The opioid antagonist can be administered systemically (e.g., orally, intravenously) and/or locally (e.g., transdermally, percutaneously). Methods of opioid antagonist administration routes include oral administration (e.g., tablets, capsules or drops), intramuscular injection, intravenous drip, subcutaneous injection, transdermal application, and endotracheal administration. Endotracheal administration is the least desirable route of administration. In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions can also be used as pharmaceutically acceptable carriers. These can be prepared according to methods known to those skilled in the art, for example, as described in U.S. Pat. No. 4,522,811.

As the data in FIGS. 1B and 2C indicate, radiation-induced production of POMC and beta-endorphin is dependent on induction of the tumor suppressor gene p53 (see e.g., Cui, R., et al., “Central Role of p53 in the Suntan Response and Pathologic Hyperpigmentation.” Cell. 2007; 128(5): 853-64). Like radiation therapy, fatigue is a major side effect of chemotherapy. Many chemotherapeutic agents administered to cancer patients induce p53 in order to cause death of tumor cells, and data suggest that increases in p53 activity can cause increased POMC and beta-endorphin production. Therefore, it is plausible that systemic beta-endorphin elevations during chemotherapy treatment play a role in chemotherapy-induced fatigue.

Therefore, an opioid antagonist, such as a beta-endorphin antagonist, can be administered to a patient experiencing fatigue related to a chemotherapy treatment that increases systemic opioid levels (e.g., beta-endorphin levels). Optionally, methods of treating radiation-induced fatigue can also include administering an opioid antagonist to a subject receiving a chemotherapeutic agent in combination with the cutaneous irradiation. The chemotherapeutic agent can be characterized by a mechanism of action that includes inducing p53 in the subject. Examples of chemotherapeutic agents include: fluorouracil (5-FU), paclitaxel, leptomycin B, doxorubicin, mitomycin C, camptothecin and roscovitine. Patients receiving chemotherapy can consistently experience fatigue, as chemotherapeutic agents act systemically, affecting more cells of the body than the more localized radiation therapy. As a result of more cells being directly drug-exposed, more cells may have up-regulated POMC and β-endorphin production. Induction of p53, in addition to triggering apoptosis, can also induce production of POMC, and in turn, β-endorphin production.

### Cutaneous Ultraviolet Irradiation

Methods of moderating the effects of endogenous opioids such as beta-endorphin accompanying cutaneous ultraviolet (UV) radiation exposure are based in part on the discovery that cutaneous irradiation can increase systemic beta-endorphin levels. In particular, increases in endogenous systemic opioid levels can be induced by ultraviolet irradiation. For example, cutaneous UV exposure can elevate systemic beta-endorphin levels in mice and rats (e.g., Examples 5-7 and 9). UV exposure in mice elevated systemic β-endorphin and caused changes in opioid-mediated phenotypes that were reversed by administration of the opiate antagonist naloxone. For UV radiation, 10 mJ/cm² is the standard erythema dose that is the minimal amount of UV radiation required to cause any redness on un-acclimated fair skin. This dose will cause no change in previously-tanned skin. 250 mJ/cm² is equivalent to 1-2 hours of exposure to ambient midday sun in Florida in July. UVA and UVB, or UVB irradiation is preferred (Gambichler, T., Bader, A., Vojvodic, M., Avermaete, A., Schenk, M., Altmeyer, P., and Hoffman, K. Plasma Levels of Opioid Peptides After Sunbed Exposures. British Journal of Dermatology. 2002; 147: 1207-1211).

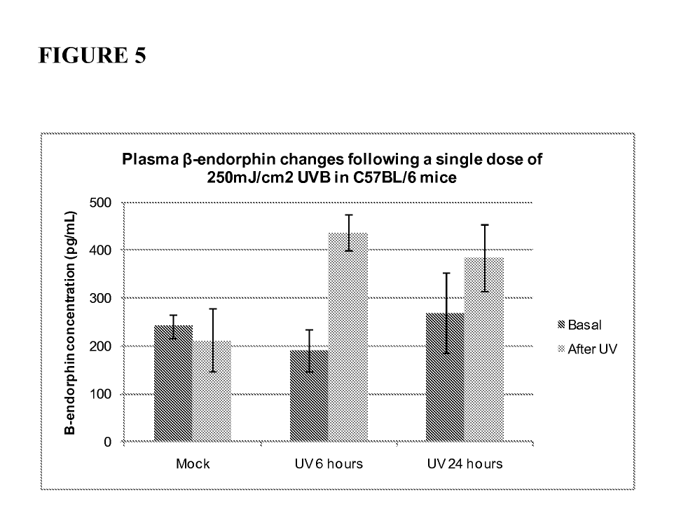

The graph in FIG. 5 demonstrates the effects of a single UV exposure on systemic beta-endorphin levels. To obtain the data shown in FIG. 5, 8-week old mice were exposed to a dose of 250 mJ/cm² or mock exposed. Blood was drawn for beta-endorphin measurement prior to, 6 hours after, and 24 hours after exposure. Data for FIG. 5 was obtained from Wild type mice that were dorsally shaved. There were 5 mice per treatment group. The data in FIG. 5 show there was a significant raise in beta-endorphin levels 6 hours after exposure which gradually diminishes 24 hours after exposure. The mock exposed mice had unusually high baseline beta-endorphin levels, and varied greatly from mouse to mouse. However, each individual mouse showed no change or a degree of decreased beta-endorphin levels post-mock exposure treatment compared to their original baseline measurement (not shown).

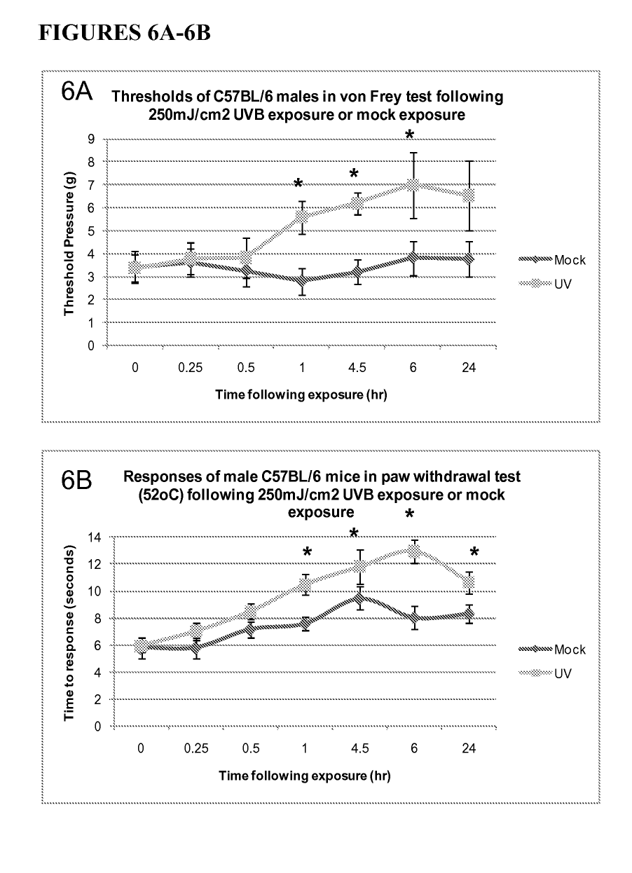

Example 6 demonstrates opioid-mediated behavioral changes in UV-exposed mice. To obtain the data shown in FIG. 6A, 8-week old mice were either exposed to a single UVB exposure, or were mock exposed, and were tested using a von Frey assay, measuring mechanical analgesia. The mice were dorsally shaved 2 days prior to taking baseline measurements. Mice were exposed to 300 mJ/cm² or mock exposed 2 days after the baseline measurements. The mice were then tested for mechanical analgesia 15 minutes, 30 minutes, 1 hour, 4.5 hours, 6 hours, and 24 hours post UVB or mock exposure. As shown in FIG. 6A, there is a large increase in mechanical analgesic threshold following UV exposure. In FIG. 6B, 8-week old mice were either exposed to a single UVB exposure, or were mock exposed, and were tested using a hot plate assay, measuring thermal analgesia. The mice were dorsally shaved 2 days prior to taking baseline measurements. Mice were exposed to 300 mJ/cm² or mock exposed 2 days after the baseline measurements. The mice were then tested for mechanical analgesia 15 minutes, 30 minutes, 1 hour, 4.5 hours, 6 hours, and 24 hours post UVB or mock exposure. Data for FIGS. 6A and 6B were obtained from male Wild type mice, with 10 mice per treatment group (respectively). An asterisk indicates p<0.05 by t-test. Although the increase in thermal analgesic threshold was not as large as in the mechanical threshold, it is still statistically significant (FIG. 6B). Analgesic thresholds peak at 6 hours and decrease at 24 hours, similarly to measured beta-endorphin levels.

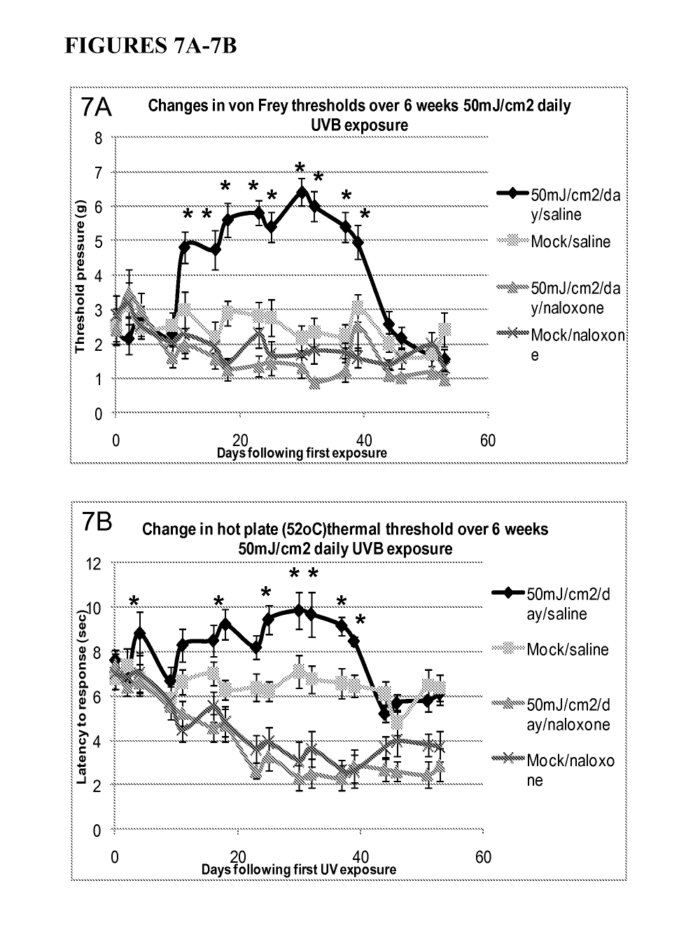

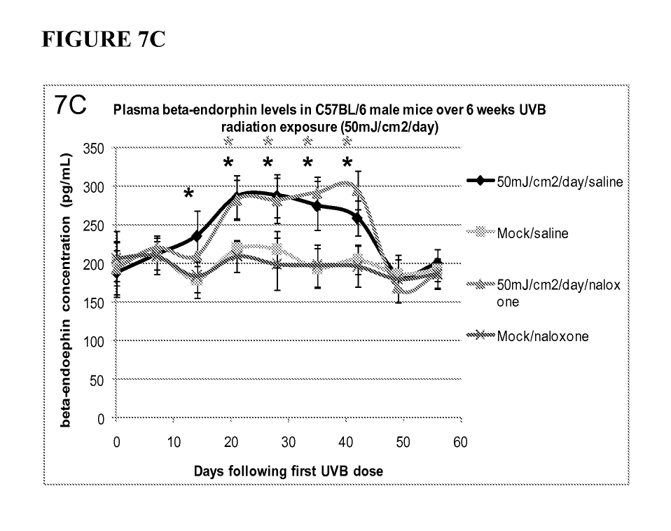

Example 7 shows UV-induced changes in systemic beta-endorphin and pain thresholds that are naloxone-reversible. To obtain the data shown in FIGS. 7A, 7B and 7C, eight-week-old male C57BL/6 mice were dorsally depilated and either exposed to 50 mJ/cm2/day or mock exposed 5 days/week for 6 weeks. Blood was drawn prior to, once weekly during, and once weekly for 2 weeks following the end of the regimen for β-endorphin measurement. In addition, pain thresholds in both the von Frey and hot plate assay were measured prior to, twice weekly during, and twice weekly for 2 weeks following the end of the regimen. One subgroup of UV-exposed mice and one subgroup of mock-treated mice were injected with naloxone (10 mg/kg, ip) and another subgroup of UV-exposed mice and mock-treated mice were injected with saline (ip) 15 minutes prior to each pain threshold testing. Pain threshold measurements were taken on 2 non-consecutive days and on a different day from the blood draws each week. FIG. 7A shows the results of the von Frey assay. FIG. 7B shows the results of the hot plate assay. Error bars indicate +/−SEM. FIG. 7C shows plasma β-endorphin levels. Error bars indicate +/− standard deviation. For all figures (*) above the graph line indicates statistical significance above baseline values for that group of p<0.05, and (*) below the graph indicates values below baseline values that are statistically significant to p<0.05 by Student's t-test.

Basal beta-endorphin in transgenic mice that over express stem cell factor in their keratinocytes was also measured. This transgene results in increased numbers of melanocytes in the epidermis, and thereby also increased tone of cutaneous MC1R signaling. These mice (called K14SCF mice) have twice the amount of basal plasma beta-endorphin compared to congenic wild type mice (FIG. 8). To obtain the data shown in FIG. 8, blood was drawn from 8-week old male mice. Plasma was obtained and beta-endorphin levels were measured. Data for FIG. 8 was obtained from Wild type (E/E) mice, K14SCF WT type mice, e/e type mice, and K14SCF e/e type mice. The K14SCF WT type mice are wild type mice that express transgenic stem cell factor which results in increased numbers of melanocytes in the epidermis. The e/e type mice are red-furred mice lacking functional MC1R. The K14SCF e/e type mice are red-furred mice that lack functional MC1R and also express the transgene stem cell factor that results in increased numbers of melanocytes in the epidermis.

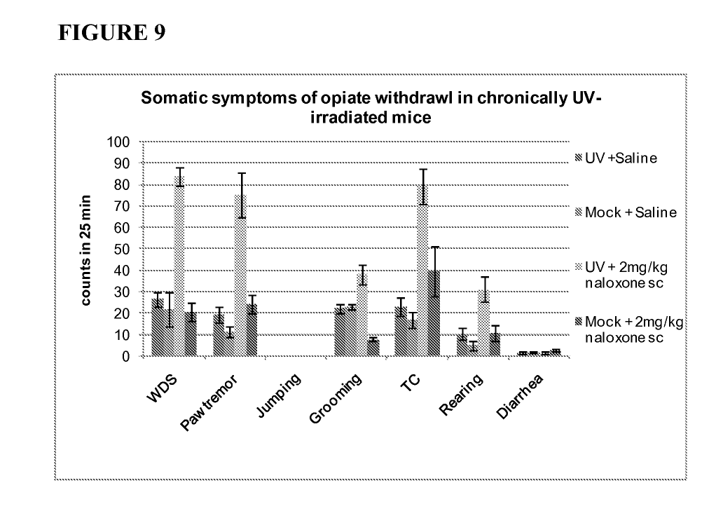

FIG. 9 shows data obtained according to Example 8, indicating somatic symptoms of withdrawal in chronically UV-irradiated mice treated acutely with naloxone. To obtain the data shown in FIG. 9, mice were either exposed to 50 mJ/cm²/day UVB radiation, or mock-exposed 5 days per week for 6 weeks. These two groups of mice were divided into subgroups and either injected with a dose of 2 mg/kg naloxone(sc) or saline. Immediately after injection, the mice were observed for somatic symptoms of opiate withdrawal for a period of 25 minutes. The symptoms observed include wet dog shake, paw tremor, jumping, grooming, teeth chatter, rearing and diarrhea.

These results, together with the published findings regarding redhead pain tolerance, suggest that basal beta-endorphin measurement may predict how well patients will respond to morphine treatment for pain (and other opiate drugs), and may also provide a diagnostic tool to identify those who are at risk for becoming addicted to opiate drugs, thereby guiding physicians' choices regarding pain management. Beta-endorphin may also be elevated in settings of fatigue other than over the course radiation therapy, including in psychiatric disorders such as major depression.

Methods of mediating the effects of cutaneous ultraviolet irradiation on endogenous opioid levels can include administering an opioid antagonist to a subject before, during or after exposure to UV cutaneous irradiation. These methods can include administering to a subject exposed to cutaneous irradiation with ultraviolet irradiation an opioid antagonist in an amount effective to reduce beta-endorphin levels in the blood of the subject to mitigate or prevent a medical condition in the subject associated with the cutaneous irradiation.

In one example, the opioid antagonist is administered in an amount to reduce the motivation of a subject to seek voluntary UV irradiation (e.g., tanning), by reducing the physical effect of increased systemic beta-endorphin. The subject can be a self-identified frequent user of ultraviolet irradiation (e.g., tanning). UV exposure is known to be a risk factor for skin cancer; and avoidance of UV can reduce risk and improve prevention. Despite widespread awareness of the link between UV exposure and skin cancer, skin cancer incidence has been increasing at a rate of 3% per year since the 1980s. One major cause could be increased UV exposure among the population, despite widespread efforts to raise awareness of the UV-skin cancer link (de Gruijl, F. R. Skin Cancer and Solar UV Radiation. European Journal of Cancer. 1999; 35(14): 2003-9. Robinson, J. K., Rigel, D. S., and Amonette, R. A. Trends in Sun Exposure Knowledge, Attitudes, and Behaviors: 1986 to 1996. Journal of the American Academy of Dermatology. 1997; 37(2 Pt 1): 179-86). One subpopulation that has been especially refractory to efforts to decrease human UV exposure for skin cancer prevention is frequent UV-seekers (defined as indoor or outdoor tanning 2 or more times/week). Frequent UV-seeking is associated with an increased risk of skin cancer, yet despite this fact, frequent UV-seekers continue to expose themselves to UV. By treating UV-seekers with opioid antagonists, this behavior can be curbed and provide a mode of skin cancer prevention in this high-risk group.

In other examples, the opioid antagonist can be administered to a subject at risk for cutaneous damage from ultraviolet irradiation, such as individuals who lack MC1R receptor signaling in melanocytes. For example, although redhead individuals (with two nonfunctional copies of the MC1R gene) can increase POMC and beta-endorphin following UV exposure, the POMC-derived alpha-MSH (the natural ligand that binds MC1R to induce pigment production in melanocytes) binds to a non-functional MC1R that cannot communicate to the melanocyte to increase pigment production. As a result, these individuals can increase production of POMC but cannot generate an appropriate response (tanning) to the peptides made from POMC. The treatment of such subjects is based in part on the indication that red-furred mice lack functional MC1R (Mogil, J. S., Ritchie, J., Smith, S. B., Strasburg, K., Kaplan, L., Wallace, M. R., Romberg, R. R., Bijl, H., Sarton, E. Y., Fillingim, R. B., and Dahan, A. Melanocortin-1 Receptor Gene Variants Affect Pain and Mu-opioid Analgesia in Mice and Humans. Journal of Medical Genetics. 2005; 42(7): 583-7). As shown in Example 1, basal beta-endorphin levels measured in both red-furred mice and in congenic wild type controls showed that the red-furred mice consistently have half the basal beta-endorphin in their plasma compared to the wild type mice.

Skin cells are believed to play a causative role in increasing endogenous beta-endorphin levels during and after cutaneous UV irradiation. In the cutaneous response to UV exposure, epidermal keratinocytes increase production of the POMC protein. POMC is posttranslationally cleaved into biologically functional peptides, which are secreted for local effect or migration to the bloodstream (Cui, R., Widlund, H. R., Feige, E., Lin, J. Y., Wilensky, D. L., Igras, V. E., D'Orazio, J., Fung, C. Y., Schanbacher, C. F., Granter, S. R., Fisher, D. E. Central Role of p53 in the Suntan Response and Pathologic Hyperpigmentation. Cell. 2007; 128(5): 853-64). One of these POMC-derived peptides is the endogenous opioid β-endorphin. In addition to their properties as pain relievers, opioids have reinforcing properties, can be addictive in certain cases, and are considered potential substances of abuse by virtue of their activity in reward centers of the brain that stimulate positive, euphoric sensations. In addition, when administered chronically, opioids can confer tolerance such that one becomes refractory to the effects of a given dose and require higher doses to achieve a desired effect, as well as physical dependence in which removal or antagonism of opioid effect results in a battery of withdrawal symptoms. Accordingly, methods to discourage frequent tanning can include administering an opioid antagonist, blocking the beta-endorphin receptor and therefore any kind of addictive behavior.

Methods of treating Seasonal Affective Disorder (SAD) are also provided. SAD is a condition in which an individual feels depressed and lethargic with a tendency to overeat, oversleep and crave carbohydrates. In its more severe forms, the affected person is totally withdrawn and unable to successfully function in society. This disorder is most commonly observed during the winter months, when skies are cloudy and overcast, with long periods of little or no natural sunlight exposure Many persons experiencing “winter blues”, or “cabin fever” are probably experiencing some lesser degree of SAD or light hunger. Most persons affected note marked relief from their symptoms of depression after exposure to sunlight, for instance during and following winter vacations to sunny climates. Methods of treating SAD can include cutaneously irradiating a subject with UV radiation to alter systemic beta-endorphin levels. One specific method of treating SAD can further include identifying individuals at risk for SAD or suffering from SAD by measuring basal beta-endorphin levels in the blood of the subjects, cutaneously irradiating the subjects, and measuring an increase in beta-endorphin levels in the blood of the subjects. Differences in basal beta-endorphin levels and/or a measured increase in blood of the subjects in response to cutaneous irradiation (e.g., UV irradiation) can be the basis for identifying individuals at risk for SAD. In addition, the method of treating SAD can include administering an opioid antagonist, such as a beta-endorphin antagonist, to a subject.

### Screening Methods

Methods of identifying compounds that mediate (e.g., block) the opioid receptors or opioid receptor binding compounds are also provided. The therapeutic dose of compounds found to counteract the effect of elevated beta-endorphin may differ depending on the magnitude of the beta-endorphin elevation (which may vary with intensity of the endorphin-elevating stimulus (e.g., radiation) or time spent exposed to the stimulus. For example, a relationship (e.g., dose-response) can be established between cutaneous irradiation at a given dose and elevated systemic beta-endorphin at a time subsequent to the cutaneous irradiation for a given subject (e.g., a mouse or other animal). Subsequently, a compound can be administered to the subject and the effect of the compound (if any) on opioid-mediated behaviors or phenotypes observed in the absence of the compound can be measured. A method for determining therapeutic doses of compounds found to be able to counteract the effect of elevated beta-endorphin are provided.

By measuring beta-endorphin levels, drugs and strategies for treating and preventing conditions such as opiate addiction and systemic effects associated with opioid fluctuations, can be developed. In addition to being a good prognostic indicator or diagnostic tool, beta-endorphin measurement can be used to evaluate how current drugs that act on the opiate axis affect endogenous opioids (e.g., findings in FIG. 3C that show tail-irradiated mice treated with naloxone prior to behavioral testing showed elevations in systemic beta-endorphin over and above the elevation caused by tail radiation alone). Methods of identifying compounds can be used to determine mechanisms of opioid-induced effects, including those resulting from drug-induced fluctuations in endogenous opioids.

Methods for identifying subjects at risk for developing radiation-induced fatigue or sun-seeking behavior are also provided. Basal beta-endorphin measurement can be used as a prognostic indicator to identify subjects most likely to be affected by stimuli that alter beta-endorphin levels. For example, differences in basal beta-endorphin levels were observed in different genetic strains of mice. These same strains of mice demonstrated differences in analgesic phenotypes mediated by differences in basal beta-endorphin. Genetic differences may also account for differences in magnitude of beta-endorphin elevation following a stimulus (e.g., radiation), and this degree of change can be used to identify subjects most at risk for developing radiation-induced fatigue or sun-seeking behavior.

### Opioid Antagonist Compositions

The opioid antagonist can be selected to block or reduce endogenous opioid receptor binding by beta-endorphin. Opioid antagonists can include cyprodime, nalmefene, naloxone, naltrexone, alvimopan, methylnaltrexone, nor-b inaltorphimine, diprenorphine, buprenorphine, cyclazocine, cyclorphan, N-methylnaloxone, ALKS 33 (Alkermes, Waltham, Mass.), ALKS 37 (Alkermes, Waltham, Mass.), 6-amino-6-desoxonaloxone, levallorphan, nalbuphine, naltrendol, naltrindole, nalorphine, oxilorphan, pentazocine, piperidine-N-alkylcarboxylate opioid antagonists (such as those described in U.S. Pat. Nos. 5,159,081; 5,250,542; 5,270,328; and 5,434,171), opioid antagonist polypeptides (such as those described by R. J. Knapp, L. K. Vaughn, and H. I. Yamamura in “The Pharmacology of Opioid Peptides”, L. F. Tseng, Ed., p. 15, Harwood Academic Publishers, (1995)), and derivatives or mixtures thereof.

The opioid antagonist can be administered before, during and/or after cutaneously irradiating the subject in a therapeutically effective dose. The dose of the opioid antagonist can be selected to reduce beta-endorphin binding to opioid receptors. For oral administration, the dose levels can be from 25 mg to 800 mg/day. For intramuscular injection, the dose levels can be from 0.4 mg/mL to 1 mg/mL. For intravenous drip, the dose levels can be from 0.25-6.25 mg/hour, or 0.1 mg/70 kg-0.5 mg/70 kg. For subcutaneous injection, the dose levels can be from 0.4 mg/mL to 1 mL. For endotracheal administration, dilute to 1-2 mL with normal saline; flush with 5 cc of saline and then administer 5 ventilations. For transdermal application, the dose levels can be about 1.8 to 2.0 mg/day.

Compositions for cutaneously delivering an opioid antagonist are also provided, such as a lotion or cream. For example, a sun screen compositions can be formulated to contain the opioid antagonist. Anti-sun/sunscreen fluid compositions are quite often in the form of an emulsion of oil-in-water type (i.e., a cosmetically acceptable support consisting of an aqueous dispersing continuous phase and of an oily dispersed discontinuous phase) that contains, in varying concentrations, one or more standard lipophilic and/or hydrophilic organic screening agents capable of selectively absorbing the harmful UV radiation, these screening agents (and the amounts thereof) being selected as a function of the desired sun protection factor, the sun protection factor (SPF) being expressed mathematically as the ratio of the dose of UV radiation required to reach the erythema-forming threshold with the UV-screening agent, to the dose of UV radiation required to reach the erythema-forming threshold without UV-screening agent.

The compositions can include a radiation absorbing compound and an opioid antagonist. For example, suitable radiation absorbing compounds include organic UV-screening agents, such as one or more compounds selected from among cinnamic derivatives; anthranilates; salicylic derivatives; dibenzoylmethane derivatives; camphor derivatives; benzophenone derivatives; .beta.,.beta.-diphenylacrylate derivatives; triazine derivatives; benzotriazole derivatives; benzoalmalonate derivatives, especially those cited in U.S. Pat. No. 5,624,663; benzimidazole derivatives; imidazolines; bis-benzoazolyl derivatives as described in EP-669,323 and U.S. Pat. No. 2,463,264; p-aminobenzoic acid (PABA) derivatives; ethylenebis-(hydroxyphenyl-benzotriazole) derivatives as described in U.S. Pat. Nos. 5,237,071, 5,166,355, GB-2,303,549, DE-197,26,184 and EP-893,119; benzoxazole derivatives as described in EP-0-832,642, EP-1,027,883, EP-1-300,137 and DE-101,62,844; screening polymers and screening silicones such as those described especially in WO 93/04665; .alpha.-alkylstyrene-derived dimers such as those described in DE-198,55,649; 4,4-diarylbutadienes such as those described in EP-0-967,200, DE-197,46,654, DE-197,55,649, EP-A-1-008,586, EP-1-133,980 and EP-133,981, merocyanin derivatives such as those described in WO 04/006878, WO 05/058269 and WO 06/032741; and mixtures thereof.

The opioid antagonist can be formulated in an aqueous composition that further comprises standard cosmetic adjuvants, including one or more materials selected from fatty substances, organic solvents, ionic or nonionic, hydrophilic or lipophilic thickeners, demulcents, humectants, opacifiers, stabilizers, emollients, silicones, antifoams, fragrances, preservatives, anionic, cationic, nonionic, zwitterionic or amphoteric surfactants, fillers, polymers, propellants, acidifying or basifying agents or any other ingredient usually used in cosmetics and/or dermatology.

Opioid antagonists can also be administered transdermally, for example to provide constant absorption into the bloodstream by way of very small capillaries found within living human tissue at prescribed constant rates, by diffusion through skin.

Unless otherwise defined, all technical and scientific terms used herein have the same meaning as commonly understood by one of ordinary skills in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety.

### REFERENCES

1. Kwan, K. Y., Allchorne, A. J., Vollrath, M. A., Christensen, A. P., Zhang, D. S., Woolf, C. J., and Corey, D. P. TRPA1 Contributes to Cold, Mechanical, and Chemical Nociception but is Not Essential for Hair-cell Transduction. Neuron. 2006; 50(2): 277-89.
2. Aravich, P. F., Rieg, T. S., Lauterio, T. J., and Doerries, L. E. Beta-endorphin and Dynorphin Abnormalities in Rats Subjected to Exercise and Restricted Feeding: Relationship to Anorexia Nervosa? Brain Research. 1993; 622(1-2): 1-8.
3. Gianoulakis, C., de Waele, J. P., and Thavundayil, J. Implication of the Endogenous Opioid System in Excessive Ethanol Consumption. Alcohol. 1996; 13(1): 19-23.
4. Petraglia, F., Bacchi Modena, A., Comitini, G., Scazzina, D., Facchinetti, F., Fiaschetti, D., Genazzani, A. D., Barletta, C., Scavo, D., and Genazzani, A. R. Plasma Beta-endorphin and Beta-lipotropin Levels Increase in Well Trained Athletes After Competition and Non Competitive Exercise. Journal of Endocrinological Investigation. 1990; 13(1): 19-23.
5. Welch, C. C., Kim, E. M., Grace, M. K., Billington, C. J., and Levine, A. S. Palatability-induced Hyperphagia Increases Hypothalamic Dynorphin Peptide and mRNA Levels. Brain Research. 1996; 721(1-2): 126-31.
6. Warthan, M. M., Uchida, T., and Wagner, R. F., Jr. UV Light Tanning as a Type of Substance-related Disorder. Archives of Dermatology. 2005; 141(8): 963-6.
7. Feldman, S. R., Liguori, A., Kucenic, M., Rapp, S. R., Fleischer, A. B., Jr., Lang, W., and Kaur, M. Ultraviolet Exposure is a Reinforcing Stimulus in Frequent Indoor Tanners. Journal of the American Academy of Dermatology; 2004; 51(1): 45-51.
8. Zeller, S., Lazovich, D., Forster, J., and Widome, R. Do Adolescent Indoor Tanners Exhibit Dependency? Journal of the American Academy of Dermatology. 2006; 54(4): 589-96.

### EXAMPLES

The invention is further described in the following examples, which do not limit the scope of the invention described in the claims. Methods of making, analyzing, and characterizing some aspects of the polymer electrolytes are described below.

### Examples 1-4

### Increasing Beta-Endorphin Levels with Ionizing Radiation

The methods and materials described below were used for Examples 1-4.

### Mice

For all experiments, mice used were in a C57BL/6 background. In all experiments wild type mice were used, and in select experiments mice containing homozygous deletion of the p53 gene (p53−/−) were used. All mice were 8 weeks old at the beginning of each experiment. For behavioral assays, only males were used, as females demonstrate slight fluctuations in behavioral responses with the estrous cycle. Blood Draws and Preparation for Radioimmunoassay Mice were placed in a standard restrainer. A 30-gauge needle was inserted into the tail vein, and 200 uL of blood was collected in EDTA microvette tubes containing 5 TIU aprotinin. Tubes were maintained on ice at all times. Tubes were spun at 3500 rpm at 4° C. for 20 minutes. Plasma was isolated for β-endorphin measurement by commercially available radioimmunoassay (Phoenix Pharmaceuticals).

### Radiation Exposure

For experiments testing the effect of total-body radiation exposure on β-endorphin levels, mice were placed in a round cage with a filter top fitted inside a GammaCell 40 Exactor irradiator (MDS Nordion). Mice received a total-body dose of 7.5 Gy (approximately 7 minutes and 30 seconds) or 9 Gy (approximately 9 minutes). Blood was drawn prior to and 24 hours following radiation exposure.

For experiments testing β-endorphin fluctuations over a regimen of daily tail radiation, mice were placed in a lead restrainer that allows tail protrusion, and their tail were exposed to 5 Gy radiation (approximately 5 minutes in the irradiator), 5 days/week for 6 weeks. Mock-treated mice were placed in the lead restrainers in the irradiator for 5 minutes with the machine off. In all experiments blood was drawn prior to the radiation regimen, once weekly during the radiation regimen and, depending on the experiment, for 2-5 weeks following the end of the regimen.

### Straub Tail Measurement

Mice undergoing tail radiation or mock treatment were assessed for Straub Tail according to a slightly modified method of Zarrindast, M. R., Ghadimi, M., Ramezani-Tehrani, B., and Sahebgharani, M. Effect of GABA Receptor Agonists or Antagonists on Morphine-induced Straub Tail in Mice. International Journal of Neuroscience. 2006; 116(8): 963-73, by which Straub tail is scored on a scale of 0-2 according to the angle of elevation of the tail from the horizontal plane (0=tail relaxed and no elevation; 1=tail is rigid and slightly elevation 1-10° from horizontal; 1.5=11-45 degree elevation with rigidity at the base of the tail; 2=46-90° elevation with rigidity at the base of the tail). For each measurement, each mouse is scored every 10 seconds for 1 minute, and the final score for each mouse at a given measurement point is the average of these 7 values. Mice undergoing tail radiation or mock treatment were scored for Straub Tail immediately following radiation exposure, 2-4 times per week starting during the second week of this experiment. There were 2 groups of mice. One group received daily tail radiation over 6 weeks followed by a 1-week break, then a 4-week regimen of mock treatment. A second group of mice received daily mock treatment over the first 6 weeks (acting as controls for the tail-irradiated mice), followed by a 1-week break and then a 4-week regimen of daily tail radiation. There were 10 mice/treatment group. After the 17th irradiation, all mice were scored as described above immediately following tail radiation. Following this, one subgroup of tail-irradiated mice was injected with 10 mg/kg naloxone (ip), and the other subgroup of tail-irradiated mice was injected with saline (ip). Straub tail was reassessed as described above at 20 minutes following naloxone injection.

### Behavioral Assays Von Frey Assay

In this assay of mechanical nociceptive threshold, mice were placed on an elevated wire mesh grid, and the plantar surface of the left hind paw was poked with fibers calibrated to different pressures. Mice were each poked 10 times at each pressure at a rate of 1/second, starting with the lowest pressure and stopping at the pressure that elicits a positive response for a given mouse, thus indicating the mechanical nociceptive threshold for that mouse. A positive response was 2/10 responses of paw flinching, fluttering, or licking immediately following the poke. These experiments were performed in mice undergoing daily tail radiation or mock tail radiation as follows.

Prior to the start of the tail radiation regimen, mice were habituated over one week by spending 1 hour/day in individual wire mesh cages on the elevated wire mesh grid. In addition, prior to each measurement, mice spent 30 minutes on the wire mesh grid to habituate before measurements were taken. Two measurements of baseline thresholds were taken on two different days prior to the start of the radiation regimen. During the radiation regimen and for 2 weeks following the end of the regimen, mice were tested twice per week on two different, non-consecutive days. Mice receiving mock treatment and 1 subgroup of mice receiving tail radiation were injected with saline (ip) 15 minutes prior to testing. A second subgroup of mice receiving daily tail radiation was injected with naloxone (10 mg/kg, ip) 15 minutes prior to behavioral testing. There were 10 mice/treatment group.

### Hot Plate Assay

In this assay of thermal nociceptive threshold, mice are placed on a metal plate thermostatically controlled to remain at 52° C. surrounded by Plexiglas walls. Thermal analgesic threshold is measured as the latency to paw flinch or flutter, paw licking, or attempt to escape (Mogil, J. S., Wilson, S. G., Bon, K., Lee, S. E., Chung, K., Raber, P., Pieper, J. O., Hain, H. S., Belknap, J. K., Hubert, L., Elmer, G. I., Chung, J. M., and Devor, M. Heritability of Nociception II. ‘Types’ of Nociception Revealed by Genetic Correlation Analysis. Pain. 1999; 80(1-2): 83-93). For these experiments, the same mice used in the von Frey assay were used in the hot plate assay, and each hot plate measurement was taken immediately following the von Frey assay. These experiments were performed in mice undergoing daily tail radiation or mock tail radiation as follows. Prior to the start of the tail radiation regimen, mice were habituated over 1 week by spending 5 minutes/day individually on the metal plate at room temperature within the Plexiglas enclosure. Two measurements of baseline thermal thresholds were taken on two different days prior to the start of the radiation regimen. During the radiation regimen and for 2 weeks following the end of the radiation regimen, measurements were taken twice per week on two different, nonconsecutive days. Mice receiving mock treatment and 1 subgroup of mice receiving daily tail radiation were injected with saline 15 minutes prior to testing. A second subgroup of mice receiving daily tail radiation was injected with 10 mg/kg naloxone 15 minutes prior to behavioral testing.

### Example 1

### Total Body Ionizing Irradiation Elevates Systemic β-Endorphin Levels in Mice

Ionizing radiation exposure results in a p53-dependent increase in plasma β-endorphin. FIG. 1A shows that in wild type C57BL/6 mice, at 24 hours following a single, total-body dose of 7.5 Gy or 9 Gy gamma radiation results in a tripling of plasma β-endorphin levels compared to basal values in the same mice.

In response to UV exposure, keratinocytes in the skin increase production of the β-endorphin precursor protein, POMC, in a p53-dependent manner. To ascertain whether the plasma β-endorphin elevation following gamma radiation exposure is also p53-dependent, the data from FIG. 1B was obtained. FIG. 1B shows that at 24 hours following a single, total-body dose of 7.5 Gy or 9 Gy gamma radiation, C57BL/6 p53−/− mice show no significant change in plasma β-endorphin levels compared to baseline values in the same mice. As in the comparison between wild type and p53−/− basal β-endorphin values in FIG. 1, the data indicates that p53−/− mice have 2-3 times greater basal levels of β-endorphin compared to wild type mice.

### Example 2

### Tail-Only Ionizing Irradiation Elevates Systemic β-Endorphin Levels in Mice

Gamma radiation can penetrate deep into the body, well beyond the skin. However, radiation-induced fatigue is often experienced by patients receiving minimally penetrating or tangential field radiation, such as breast cancer patients. This suggests that factors produced in the skin play a causative role. The fact that β-endorphin is produced in the skin in response to UV exposure, along with the fact that lethargy and sedation are common side-effects of exogenous systemic opioid administration make β-endorphin an attractive candidate for contributing to radiation-induced fatigue. Having established that total-body gamma radiation exposure results in systemic elevations in β-endorphin, further tests were performed to determine whether gamma radiation exposure to the skin alone can cause elevations in systemic β-endorphin. For these experiments, low to moderate daily tail radiation were used in mice to model a radiation therapy regimen for cancer patients. FIGS. 2A and 2B show that systemic β-endorphin levels are responsive to a daily tail radiation regimen in both female (FIG. 2A) and male (FIG. 2B) mice. Mice receiving 5 Gy/day tail radiation for 6 weeks followed by mock treatment for 4 weeks show β-endorphin levels that rise significantly by week 2 of the radiation regimen, peak at week 4 of the regimen, and begin to dip in week 5. Over the following 4 weeks of mock treatment, β-endorphin levels stabilize at levels lower than levels achieved during the radiation regimen. Mock treated controls over the first 6 weeks of treatment show stable levels of β-endorphin during the mock treatment regimen. However, when these mice begin a 4-week regimen of 5 Gy/day tail radiation following the 6 weeks of mock treatment, β-endorphin levels rise significantly by week 2 into the true radiation treatment.

Additional tests were performed to determine whether this systemic increase in β-endorphin over a tail radiation regimen is p53-dependent. FIG. 2C shows that in p53−/− mice, there is no significant β-endorphin over a 3-week regimen of daily tail radiation plus 1 week following the end of the regimen, indicating that β-endorphin elevation seen in wild type mice is a p53-dependent phenomenon. After 3 weeks of the tail radiation regimen, these mice began to develop tumors on their tails.

A reproducible dip in systemic β-endorphin levels and analgesic responses was observed from peak levels prior to the end of the 6-week radiation regimen, usually at weeks 5 and 6. Towards the end of the 6-week radiation regimen, the mouse tail becomes fibrotic and the most superficial cells, the keratinocytes, begin to slough off. Since keratinocytes are the primary skin cells that increase POMC production following radiation exposure (Cui, R., Widlund, H. R., Feige, E., Lin, J. Y., Wilensky, D. L., Igras, V. E., D'Orazio, J., Fung, C. Y., Schanbacher, C. F., Granter, S. R., Fisher, D. E. Central Role of p53 in the Suntan Response and Pathologic Hyperpigmentation. Cell. 2007; 128(5): 853-64), it is most likely that their loss results in less total POMC production following radiation exposure to a degree that is physiologically significant.

### Example 3

### Naloxone-Reversible Straub Tail Over a Regimen of Tail-Only Radiation Exposure

Straub tail in mice is a stiffening and elevation of the tail that results from opioid-mediated contraction of the sacrococcygeus dorsalis muscle at the base of the tail. This phenotype has been demonstrated in morphine-treated mice (Belknap, J. K., Noordewier, B., and Lame, M. Genetic Dissociation of Multiple Morphine Effects Among C57BL/6J, DBA/2J and C3H/HeJ Inbred Mouse Strains. Physiology & Behavior. 1989; 46(1): 69-74), and is reversible by treatment with opioid antagonists, such as naloxone (Zarrindast, M. R., Ghadimi, M., Ramezani-Tehrani, B., and Sahebgharani, M. Effect of GABA Receptor Agonists or Antagonists on Morphine-induced Straub Tail in Mice. International Journal of Neuroscience. 2006; 116(8): 963-73).

To test whether the systemic elevation of β-endorphin is significant enough to cause changes in opioid-mediated phenotypes, Straub tail measurements were taken in wild type mice over the course of a 6-week regimen of daily tail radiation. One group of mice (FIG. 3A) was treated with 6 weeks of daily tail radiation followed by a 1-week break and 4 weeks of mock exposure. A second group of mice (FIG. 3B) began with 6 weeks of mock treatment, followed by a 1-week break and 4 weeks of daily tail radiation. Straub Tail was measured from weeks 2-11 in both groups, and mock treated groups served as controls to which to compare tail-irradiated mice. Previous studies have demonstrated no difference in Straub Tail phenotype between mock treated and tail-irradiated mice over the first week of radiation treatment. Accordingly, this experiment measured Straub Tail in the second week of radiation treatment, comparing treated and mock treated mice (FIGS. 3A and 3B). As shown in FIGS. 3A and 3B, the Straub Tail phenotype occurs in response to tail radiation exposure over the 6-week regimen and returns to basal values following the radiation regimen with kinetics that approximately parallel plasma β-endorphin fluctuations. To test whether the observed Straub Tail is reversible by opioid antagonists, the tail-irradiated mice were divided into two subgroups following the 17th tail irradiation. For all mock-treated and tail-irradiated mice, Straub Tail were measured immediately following treatment. Then, for one subgroup of tail-irradiated mice, the opioid antagonist naloxone (10 mg/kg, ip) was administered and for the other subgroup saline was administered. Twenty minutes following these injections, Straub Tail were measured again. FIG. 3C shows that 20 minutes following naloxone injection, Straub Tail has been abolished to near mock-treated levels, but saline injection has no effect on the Straub Tail phenotype. FIG. 3D shows representative photographs of mice from each treatment group.

### Example 4

### Naloxone Reversible Changes in Analgesic Responses Over the Course of Tail-Only Radiation Exposure

Another set of phenotypes that assess opioid effect in mice are changes in analgesic response. To assess whether other opioid-mediated phenotypes are altered in mice over the course of tail radiation treatment, changes in mechanical and thermal analgesic responses were tracked during a 6-week regimen and for 2 weeks following the end of the regimen in 4 groups of mice: a tail-irradiated group that received a 10 mg/kg injection of naloxone 15 minutes prior to analgesic testing, a tail-irradiated group that received a saline injection 15 minutes before analgesic testing, a mock treated control group that received a saline injection 15 minutes before testing, and a mock-treated control group that received 10 mg/kg naloxone 15 minutes prior to analgesic testing. For all groups of mice, blood was drawn once per week for β-endorphin measurement. It appears that both mechanical (FIG. 4A) and thermal (FIG. 4B) analgesic thresholds increase over the course of the radiation in a way that approximately parallels plasma β-endorphin fluctuations (FIG. 4C) whereas mock treated mice remain more stable in both analgesic thresholds and plasma β-endorphin. In the tail-irradiated group of mice, both plasma β-endorphin and analgesic thresholds return to near baseline levels over the 2 weeks following the end of the radiation regimen. Furthermore, the changes observed in tail-irradiated mice are prevented by pretreatment with naloxone, despite significant elevation in plasma β-endorphin in these mice over the course of the radiation regimen.

Of note is that the tail-irradiated, naloxone-treated group exhibited the most dramatic increase in plasma β-endorphin over the radiation regimen, and demonstrated increased sensitivity to both mechanical and thermal stimuli as the radiation regimen progressed. This suggests the possibility that over the radiation regimen as β-endorphin elevation persists, these mice become physically dependent on the elevated β-endorphin and naloxone treatment acutely causes a withdrawal-associated hyperalgesia. However, upon naloxone injection in these mice, other phenotypes associated withdrawal (i.e., jumping, wet-dog shaking, teeth chattering, and diarrhea) were not observed acutely. Interestingly, the mock-exposed, naloxone-treated group showed no increase in plasma beta-endorphin levels, however, these mice did show increased sensitivity to mechanical and thermal stimuli compared to mock-exposed, saline-treated mice. This suggests a role for beta-endorphin in regulating basal analgesic threshold in mice.

### Examples 5-7

### Increasing Beta-Endorphin Levels with Ultraviolet Radiation

The methods and materials described below were used for Examples 5-7. Mice were obtained from the source described in Examples 1-4, however only wild type mice were used in these experiments.

### UV Radiation Exposure

All mice were dorsally shaved 2 days prior to radiation exposure. For experiments testing β-endorphin fluctuations following a single dose of UV, mice were either mock treated or exposed to 250 mJ/cm² or 300 mJ/cm² UVB, which is equivalent to the dose received over 1-2 hours of exposure to ambient midday sun in Florida in July (D'Orazio, J. A., Nobuhisa, T., Cui, R., Arya, M., Spry, M., Wakamatsu, K., Igras, V., Kunisada, T., Granter, S. R., Nishimura, E. K., Ito, S., and Fisher, D. E. Topical Drug Rescue Strategy and Skin Protection Based on the Role of Mc1r in UV-induced Tanning Nature. 2006; 443(7109): 340-4). All mice had blood drawn prior to the start of radiation. One group of exposed mice had blood drawn at 6 hours post-exposure, and a second group had blood drawn at 24 hours post-exposure. Mock-treated mice had blood drawn 14 hours post mock exposure. For low-dose daily UV exposure, mice were shaved 2 days prior to the start of the regimen and at the end of weeks 2 and 4 during the 5-week daily UVB exposure regimen. Mice were either mock exposed, or exposed to 20 mJ/cm²/day, 50 mJ/cm²/day, or 50 mJ/cm² every other day. Blood was drawn prior to the start of the radiation regimen, once weekly during the 5-week regimen. Blood draws always occurred in the morning before UV exposure for that day.

### Behavioral Assays

Mice were habituated to the von Frey elevated wire mesh grid and to the thermostatically-controlled plate used for the hot plate test as described above. Mice were shaved 2 days prior to taking 2 baseline responses in the von Frey and Hot plate assays on 2 days separated by 48 hours. Forty-eight hours following the second baseline measurement, mice were exposed to a single dose of 300 mJ/cm² UVB or mock exposure. Behavioral responses were measured in the von Frey and hot plate tests at 15 minutes, 30 minutes, 1 hour, 4.5 hours, 6 hours, and 24 hours following UV exposure.

### Example 5

### B-Endorphin Fluctuations Following a Single Acute Dose of UVB

To determine whether a single UV exposure can elevate systemic β-endorphin levels, a single dose of 250 mJ/cm² was tested to determine if this dose can cause an elevation in systemic β-endorphin. FIG. 5 shows that in wild type mice exposed to UV, there is a significant elevation of systemic β-endorphin that diminishes by 24 hours. In this experiment the basal β-endorphin levels of the mock-treated group were unusually high and the error bars are wide, thus it is difficult to tell whether no change in β-endorphin levels is detected due to the unusually high baselines or whether there really is no significant change in β-endorphin from baseline levels. Although basal levels carried significantly from mouse to mouse, each individual mouse either showed no change or a degree of decreased β-endorphin levels at the post-mock-exposure measurement compared to their own basal levels.

### Example 6

### Opioid-Mediated Behavioral Changes Following a Single Dose of UVB

It appeared that there is some degree of increase in systemic β-endorphin over the hours following UVB exposure. An evaluation of whether opioid-mediated behavioral changes occur in UVB-exposed mice over a similar timeline found a significant increase in mechanical pain threshold, as tested in the von Frey assay, in mice following a single dose of 300 mJ/cm² UVB (FIG. 6A). Changes in thermal analgesic response following UVB, although not as robust as the changes in mechanical analgesic thresholds, were present and statistically significant (FIG. 6B). Both mechanical and thermal analgesic responses changed in parallel with changes observed in β-endorphin blood levels, peaking at 6 hours and beginning to decrease at 24 hours.

### Example 7

### Changes in Systemic β-Endorphin and Pain Thresholds Over the Course of a Regimen of Daily Low-Dose UVB Exposure

This example demonstrates that UV-induced changes in pain thresholds are naloxone-reversible. Eight-week-old male C57BL/6 mice were dorsally depilated and either exposed to 50 mJ/cm2/day or mock exposed 5 days/week for 6 weeks. Blood was drawn prior to, once weekly during, and once weekly for 2 weeks following the end of the regimen for β-endorphin measurement. In addition, pain thresholds in both the von Frey and hot plate assay were measured prior to, twice weekly during, and twice weekly for 2 weeks following the end of the regimen. One subgroup of UV-exposed mice and one subgroup of mock-treated mice were injected with naloxone (10 mg/kg, ip) and another subgroup of UV-exposed mice and mock-treated mice were injected with saline (ip) 15 minutes prior to each pain threshold testing. Pain threshold measurements were taken on 2 non-consecutive days and on a different day from the blood draws each week.

### Example 8

### Somatic Symptoms of Withdrawal in Chronically UV-Irradiated Mice Treated Acutely with Naloxone

This example assesses physical dependence to opiate agents by evaluating withdrawal symptoms in mice upon acute administration of an opiate antagonist. In mice, symptoms of opiate withdrawal include shaking vigorously (called a “wet-dog shake”), paw tremors, teeth chattering, jumping, rearing, increased grooming, and diarrhea. The occurrence of these symptoms were measured in mice either exposed to 50 mJ/cm²/day UVB radiation 5 days per week for 6 weeks or mock-exposed on the same schedule. Results are shown in FIG. 9. One subgroup of each group of mice was injected with 2 mg/kg subcutaneous naloxone and number of occurrences of each of the above-mentioned symptoms was counted over 25 minutes immediately following naloxone injection. The other subgroup was administered a subcutaneous injection of saline before the 25-minute observation period. FIG. 9 shows that the UV-exposed, naloxone-treated group showed significantly more wet dog shakes, teeth chattering, paw tremoring, grooming, and rearing compared to all other groups. In addition, the UV-exposed, naloxone-treated group was the only group to show any jumping behavior at all. These results suggest that chronic UVB exposure appears to elicit a characteristic opioid physical dependence that may be mediated by increases in plasma levels of the endogenous opioid beta-endorphin over the course of the UVB exposure regimen.

### Example 9

### Induction of Naloxone-Reversible Lethargy in Rats by a Regimen of Daily Tail Radiation

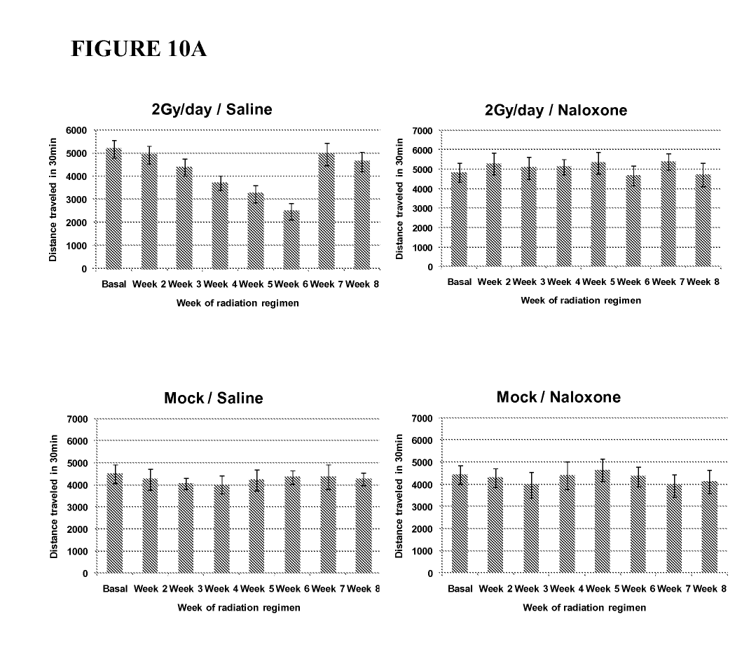

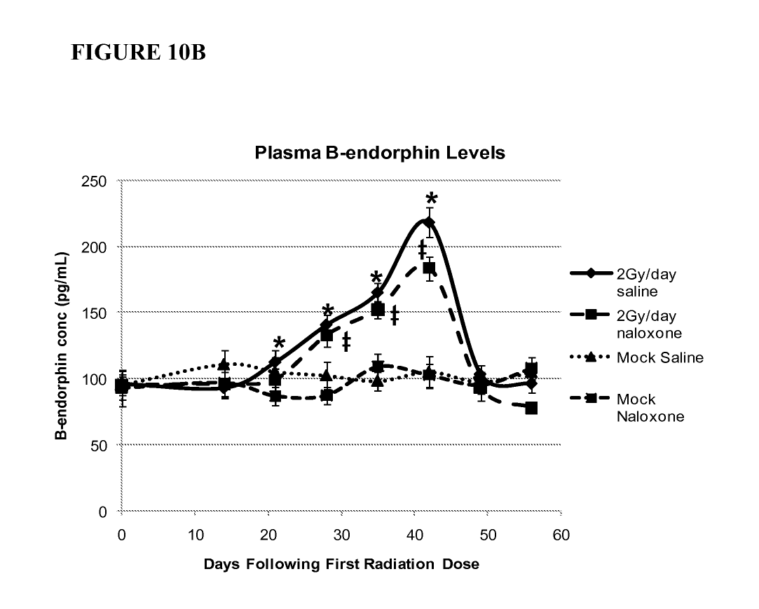

Eight week old male Sprague-Dawley rats underwent a 6-week regimen of 2 Gy/day daily tail radiation of mock exposure. Prior to the start of the regimen, baseline activity was recorded as total distance traveled in 30 minutes in the actimetry chamber. During the regimen and for 2 weeks following the end of the regimen, actimetry measurements were made once per week, and for these measurements, half of the tail-irradiated rats and half of the mock-exposed rats were injected with naloxone (1 mg/kg, ip) 15 minutes prior to actimetry testing, and the other half of tail-irradiated and mock-exposed animals were injected with saline (ip) 15 minutes prior to actimetry testing. In addition, blood was drawn prior to, once weekly during, and once weekly for 2 weeks following the end of the regimen for β-endorphin measurement. FIG. 10A shows the actimetry results. Top graphs show results for tail-irradiated rats, bottom graphs show results for mock-exposed controls. Left side graphs show results for rats injected with saline prior to actimetry testing, right side graphs show results for rats injected with naloxone prior to actimetry testing. FIG. 10B shows plasma levels of β-endorphin over the course of the 8-week monitoring period. n=9-10 rats/treatment group. Error bars indicate +/−SEM, and (*) indicates p<0.05 by two-tailed Student's t-test with respect to baseline values for that group.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. (canceled)

2. A method of treating or preventing fatigue associated with exposure to cutaneous irradiation in a subject, the method comprising
identifying a subject who has been or will be exposed to cutaneous irradiation; and
administering to the subject a therapeutically effective amount of an opiate antagonist to treat or prevent fatigue in the subject.

3. (canceled)

4. A method of treating or preventing fatigue associated with administration of a chemotherapeutic agent in a subject, the method comprising
identifying a subject who has or will receive a chemotherapeutic agent; and
administering to the subject a therapeutically effective amount of an opiate antagonist to treat or prevent fatigue in the subject.

5. The method of claim 2, wherein the cutaneous irradiation is gamma or ultraviolet cutaneous irradiation.

6. The method of claim 2, wherein the subject has been or will be exposed to cutaneous irradiation in a dose effective to increase the level of beta-endorphin in the blood of the subject.

7. The method of claim 4, wherein the subject has been or will be administered a chemotherapeutic agent that induces p53 in the subject.

8. The method of claim 2, wherein the subject has been or will be exposed to cutaneous irradiation administered at a greater dose of irradiation to a dermal surface than underlying tissue.

9. The method of claim 2, wherein the opioid antagonist is administered after the subject is exposed to cutaneous irradiation.

10. The method of claim 2, wherein the cutaneous irradiation is for therapeutic purposes

11. The method of claim 4, wherein the opioid antagonist is administered after the subject is administered the chemotherapeutic agent.

12. The method of claim 2, wherein the therapeutically effective amount of the opioid antagonist reduces an elevation of beta-endorphin level in the blood of the subject induced by the cutaneous irradiation.

13. The method of claim 2, wherein the opioid antagonist is administered systemically to the subject.

14. The method of claim 2, wherein the opioid antagonist is a μ-opioid receptor antagonist.

15. The method of claim 2, wherein the opioid antagonist is naloxone.

16. The method of claim 4, wherein the therapeutically effective amount of the opioid antagonist reduces an elevation of beta-endorphin level in the blood of the subject induced by the cutaneous irradiation.

17. The method of claim 4, wherein the opioid antagonist is administered systemically to the subject.

18. The method of claim 4, wherein the opioid antagonist is a μ-opioid receptor antagonist.

19. The method of claim 4, wherein the opioid antagonist is naloxone.
